(19) United States
(12) Patent Application Publication
(10) Pub. No.: US 2007/0249628 A1
(43) Pub. Date: Oct. 25, 2007

(54) Use of Mast Cells Inhibitors for Treating Patients Exposed to Chemical or Biological Weapons

(76) Inventors: Alain Moussy, Paris (FR); Jean-Pierre Kinet, Lexington, MA (US)

Correspondence Address: FOLEY AND LARDNER LLP;SUITE 500, 3000 K STREET NW, WASHINGTON, DC, 20007, US

(21) Appl. No.: 11/596,374

(22) PCT Filed: Apr. 19, 2005
(86) PCT No.: PCT/IB05/01459

§ 371 Date: Nov. 14, 2006

(30) Foreign Application Priority Data
May 18, 2004 (US) 10847363

(51) Int. Cl. A01N 43/54; A61K 31/404; A61K 31/421; A61K 31/426; A61K 31/517; G01N 33/566

(52) U.S. Cl. 514/256; 435/7.8; 514/266.1; 514/370; 514/377; 514/418

(57) ABSTRACT

The present invention relates to a method for treating patients exposed to chemical or biological weapons comprising administering a compound capable of depleting mast cells or a compound inhibiting mast cells degranulation, to a human in need of such treatment. Such compounds can be chosen from c-kit inhibitors and more particularly non-toxic, selective and potent c-kit inhibitors. Preferably, said inhibitor is unable to promote death of IL-3 dependent cells cultured in presence of IL-3.

The present invention relates to a method for treating patients exposed to chemical or biological weapons comprising administering a compound capable of depleting mast cells or a compound inhibiting mast cells degranulation, to a human in need of such treatment. Such compounds can be chosen from c-kit inhibitors and more particularly non-toxic, selective and potent c-kit inhibitors. Preferably, said inhibitor is unable to promote death of IL-3 dependent cells cultured in presence of IL-3.

Since 911 events, threats have materialized in different forms of terrorist attacks, and it appears that the risk might spread overtime and overseas. Measures are being taken to organize and planned protective actions for the population. Nevertheless, as of today, there is no antidote which would rapidly counteract chemical or biological weapons exposure.

For example, the toxicity of the chemical warfare blistering agent sulfur mustard (2,2′-dichlorodiethyl sulfide; SM or HD) has been investigated for nearly a century; however, the toxicological mechanisms of SM remain obscure and no antidote exists. The similarity of dermal-epidermal separation caused by SM exposure, proteolysis, and certain bullous diseases has fostered the hypothesis that SM vesication involves proteolysis and/or inflammation. Compound screening conducted by the US Army Medical Research Institute of Chemical Defense established that topical application of three tested serine protease inhibitors could reduce SM toxicity in the mouse ear vesicant model. Although most of the drugs with efficacy for SM toxicity in rodent models are anti-inflammatory compounds, no in vitro assay is in current use for screening of potential anti-inflammatory SM antidotes. IL-8 is a potent neutrophil chemotactic cytokine that is increased in human epidermal keratinocyte (HEK) cell cultures following exposure to SM and has been proposed as a marker for SM-induced inflammation (Cowan, 2002). It has been proposed by Dachir and al (Dachir, 2002) that anti-inflammatory drugs could significantly diminish HD-induced inflammation as long as the treatment is applied during the early stages following exposure.

But, in spite of several decades of research, no effective treatment to skin injuries following exposure to sulfur mustard (HD) has yet been found.

Despite the contrasts in chemistry and toxicity, for blister and nerve chemical warfare agents, there may be some analogous proteolytic and inflammatory mediators and pathological pathways that can be pharmacological targets for a single-drug multi-threat medical countermeasure. The dermal-epidermal separation caused by proteases and bullous diseases compared with that observed following exposure to the blister agent sulfur mustard (2,2′-dichlorodiethyl sulfide) has fostered the hypothesis that sulfur mustard vesication involves proteolysis and inflammation. In conjunction with the paramount toxicological event of cholinergic crisis that causes acute toxicity and precipitates neuronal degeneration, both anaphylactoid reactions and pathological proteolytic activity have been reported in nerve-agent-intoxicated animals. Two classes of drugs have already demonstrated multi-threat activity for both nerve and blister agents. Serine protease inhibitors can prolong the survival of animals intoxicated with the nerve agent soman and can also protect against vesication caused by the blister agent sulfur mustard. Poly (ADP-ribose) polymerase (PARP) inhibitors can reduce both soman-induced neuronal degeneration and sulfur-mustard-induced epidermal necrosis. Protease and PARP inhibitors, like many of the other countermeasures for blister and nerve agents, have potent primary or secondary anti-inflammatory pharmacology. It has been hypothesized that drugs with anti-inflammatory actions against either nerve or blister agent might also display multi-threat efficacy for the inflammatory pathogenesis of both classes of chemical warfare agents. (Cowan, 2003).

To face an attack efficiently, two criteria should be regarded as essentials: first, the treatment should be administered very quickly to the exposed population even in case the exact nature of the toxic compounds is not yet confirmed, second because a countermeasure is to be implemented in a small time frame, it has to be safe, it must have a broad spectrum of activity, and exert an strong antidote activity.

Therefore, the problem is to find compounds that exert such safe, strong and broad anti-inflammatory activity which could be administered to the afflicted population in the best time frame possible.

Bacillus anthracis is also classified as a serious potential threat. This etiologic agent responsible for Anthrax, is a large (1 to 1.5 μm by 4 to 10 μm), square-ended, non motile, aerobic, Gram-positive rod, with a centrally located spore. On Gram's stain preparations, the spore appears as unstained areas. In vitro, the cells frequently occur in long chains giving them a bamboo appearance. The chains of virulent forms of the bacteria are usually surrounded by a capsule. Because spores survive for many years in arid and semiarid environments (Jedrzejas, 2003) and since they are highly resistant to drying, Anthrax could be develop or transported without the need of advanced technology.

In addition, infection with Bacillus anthracis can lead to septicemia, tissue necrosis, multiple organ failure, and death (Vautier, 2003; Erickson, 2003). The endospores of Anthrax, a gram-positive bacillus found in the soil, are resistant to heat, drying, ultraviolet and gamma radiation, and many disinfectants (Erickson, 2003). Endospores are produced when deleterious conditions exist; they can survive for decades in the environment and are adaptable to being aerosolized. Anthrax infection is considered a rare event, but it has been implicated in several outbreaks, including 25 cutaneous infections caused by a single cow in Paraguay in 1987 and thousands of infections in Zimbabwe in the early 1980s (Doganay, 1983). However, because of its propensity to be used as a weapon of disease and death, it has attracted much attention in recent years (Dybowska, 2003).

Exposure to Anthrax can occur after contact with infected animals or humans via abrasions or through inhalation, ingestion, or contact with the skin (Biederbick, 2002). When exposure is cutaneous, infection is generally curable and rarely fatal (Celia, 2202). Cases of gastrointestinal exposure, such as eating infected meat, are extremely rare (Furowicz, 1999). Inhalation exposure among slaughterhouse and textile workers is somewhat more frequent. However, this has been managed effectively by immunization (Jefferson, 2000). When B. anthracis is released in an aerosol form, the spores enter the pulmonary macrophages, which carry the organism to the lymph nodes and other suitable environments for its growth. A capsule, various proteins, and toxins are produced by the organism; the Anthrax toxin can cause septicemia, tissue necrosis, multiorgan failure, and death (Smith, 2002; Cullamar, 2002). Symptoms of Anthrax infection include fever, malaise, cough, and respiratory distress; if untreated, shock and death can occur within 36 h (Henry, 2001).

The principal virulence factors of B anthracis are capsular polypeptide (Jedrzejas, 2002) and Anthrax toxin (Bradley, 2003). The B anthracis capsule, which consists of poly-D-glutamic acid, is thought to confer resistance to phagocytosis. Anthrax toxin consists of three proteins called protective antigen (PA), edema factor (EF), and lethal factor (LF) (Ascenzi, 2002; Morourez, 2002). The major virulence genes of B anthracis have been cloned. They are found on two large plasmids, pXO1 and pXO2. pXO1, which is 184 kilobases in size, contains the genes that produce Anthrax toxin complex and their transcriptional regulators; pXO2 is 97 kilobases in size, featuring the genes responsible for capsule synthesis. The large nature of the plasmids suggests that there are perhaps other pathogenecity genes yet to be identified. The presence of both plasmids is required for virulence (Bhatnagar, 2001; Brossier, 2001).

PA, so named for its ability to provide experimental protective immunity against B anthracis, is considered the central component of Anthrax toxin. PA is an 83-kd protein that binds to target cell receptors. A small 20-kd N-terminal fragment is proteolytically cleaved from it, thereby allowing the larger cell-bound PA fragment to act as a membrane channel. EF and LF bind to exposed sites on the PA fragment and form edema toxin and lethal toxin. PA then transfers these enzymatic proteins across cell membranes and releases them into the cell cytoplasm where they exert their effects (Ascenzi, 2002).

EF is a calmodulin-dependent adenyl cyclase that converts adenosine triphosphate to cyclic adenosine monophosphate (cAMP). Thus, intracellular levels of cAMP increase and lead to the edema often seen in Anthrax Edema toxin also plays a role in inhibiting both phagocytic and oxidative burst activities of polymorphonuclear leukocytes. Generally, bacterial toxins that are capable of increasing cAMP tend to decrease the immune response of phagocytes, thereby contributing to the development of infection (Duesbery, 1999).

The action of LF continues to be a matter of study. At high concentrations, LF has been shown to cause lysis of macrophages; at lower concentrations, it may play a role in the increased expressions of tumor necrosis factor (TNF) and interleukin-1 (IL-1) (Lacy, 2002). This observation has lead to the interesting theory that TNF-α (a mediator known for its deleterious effects during septic shock) and/or other proinflammatory mediators are stored within the macrophage early in the course of Anthrax infection, when toxin levels are lower than the critical concentration required for lysis. Later, as the infection progresses and the number of bacteria increases, a threshold for lysis is reached and large amounts of preformed mediators are released in the circulation. This rapid release of inflammatory mediators may account for the sudden death seen in Anthrax victims. Data supporting the role of IL-1 and TNF were provided by Hanna et al (Hanna, 2001; Hanna, 1998; Hanna, 1999) who reported that antibodies to TNF-α and IL-1 were protective against a lethal dose of Anthrax toxin in mice.

Antibiotics and supportive care in an intensive care setting are the mainstay of therapy. Antitoxin used in the Sverdlovsk epidemic is no longer available for human use. The Anthrax bacillus is highly susceptible to penicillin, amoxicillin, chloramphenicol, doxycycline, erythromycin, streptomycin, and ciprofloxacin, but resistant to third-generation cephalosporins (Yetman, 2002; Aizenstien, 2002).

Penicillin resistance is rare in naturally occurring strains. However, it is possible to manufacture resistant strains, which is a matter of great concern in the event of biological warfare (Bryskier, 2002). Penicillin G, 4 million units every 4 h; ciprofloxacin, 400 mg every 12 h; or doxycycline, 100 mg every 12 h, are dosages often used in the treatment of inhalational Anthrax.

One problem with Anthrax infection is not the growth of the bacillus anthracis itself but the synthesis and release of Anthrax toxin that is responsible for morbidity and mortality and against which there is no antidote.

Furthermore, the extension of the infection into the body remains without clinical signs until the amount of Anthrax toxin released has caused irreversible tissue damages. At this time antibiotic therapy might not be able to counteract these pro-inflammatory related tissue damages.

As a consequence, the above mentioned need for a countermeasure which is safe, broad, and efficient is relevant for the populations suspected to have been in contact with or exposed to Anthrax. It is also the case for other potential bacteriological weapons. Again, preventive treatment should be implemented rapidly to avoid irreversible tissue damages.

To respond to the above mentioned problems, we propose to inhibit mast cells and we have developed compounds capable of depleting mast cells as well as inhibiting their activity. Our preferred compounds described hereinafter turn out to be safe while displaying a very broad activity preventing tissue damages and ultimately morbidity.

Mast cells (MC) are tissue elements derived from a particular subset of hematopoietic stem cells that express CD34, c-kit and CD13 antigens (Kirshenbaum, 1999 and Ishizaka, 1993). Immature MC progenitors circulate in the bloodstream and differentiate in tissues. These differentiation and proliferation processes are under the influence of cytokines, one of utmost importance being Stem Cell Factor (SCF), also termed Kit ligand (KL), Steel factor (SL) or Mast Cell Growth Factor (MCGF). SCF receptor is encoded by the protooncogene c-kit, that belongs to type III receptor tyrosine kinase subfamily (Boissan, 2000). This receptor is also expressed on others hematopoietic or non hematopoietic cells. Ligation of c-kit receptor by SCF induces its dimerization followed by its transphosphorylation, leading to the recruitment and activation of various intracytoplasmic substrates. These activated substrates induce multiple intracellular signaling pathways responsible for cell proliferation and activation (Boissan, 2000). Mast cells are characterized by their heterogeneity, not only regarding tissue location and structure but also at the functional and histochemical levels (Aldenborg, 1994; Bradding, 1995; Irani, 1991, 1989 and Welle, 1997).

Mast cells (MCs) are multifunctional effector cells of the innate immune system ubiquitously distributed among the tissues. Interestingly, mature MCs are distributed throughout connective or mucosal tissues, where they interface with the external environment. This preferential location of MCs at the portals of entry of many extraneous agents ensures their early contact with these external aggressors.

“Normal” MC activation is followed by the controlled release a variety of mediators that are essential for the defense of the organism against invading pathogens.

By contrast, we have found that in case of hyperactivation of MCs, uncontrolled hypersecretion of these mediators is deleterious for the body. Indeed, mast cells produce a large variety of mediators categorized into three groups: preformed granule-associated mediators (histamine, proteoglycans, and neutral proteases), lipid-derived mediators (prostaglandins, thromboxanes and leucotrienes), and various cytokines (IL-1, IL-2, IL-3, IL-4, IL-5, IL-6, IL-8, TNF-α, GM-CSF, MIP-1α, MIP-1β and IFN-γ), most of them having strong pro-inflammatory activities. For instance, a massive release of MCs mediators is responsible for anaphylactic reactions that could be sometimes fatal to the patients and are always responsible for a significant morbidity. Since MCs are distributed in almost all the body sites, hypersecretion of mediators by activated elements can lead to multiple organ failures.

Thus, MCs are not only involved in allergic reactions but also in the first steps of reaction towards a variety of infectious agents (such as most of the bacteria) or a number of natural or human-made chemical agents, such as bacterial toxins, hydrocarbons, pesticides, heavy metal, vesicant, etc.

In connection with the present invention, we have discovered that mast cells inhibitors such as c-kit inhibitors, which are capable of depleting mast and preventing degranulation, counteract the detrimental and often lethal effects of inflammation and tissue destruction induced by exposure to chemical or biological weapons. The broad range of action of mast cells inhibitors, their safety and potency allows a rapid administration after exposure. This is particularly important to minimize as much as possible damages to vital organs and morbidity. In addition, it offers the possibility of a broad and rapid anti-inflammatory treatment during a crisis where the exact nature of the attack or exposure is yet not confirmed.

A new route for treating patients exposed to chemical or biological toxic or lethal entities is provided, which consists of administering mast cells inhibitors, more particularly c-kit inhibitors.

### DESCRIPTION

The present invention relates to a method for treating patients exposed to chemical or biological weapons comprising administering a compound capable of depleting mast cells or blocking mast cells degranulation to a human in need of such treatment.

Said method for treating patients exposed to chemical or biological weapons can comprise administering a c-kit inhibitor to a human in need of such treatment. Alternatively or concurrently, it may also consist of administering an antihistamine compound or a compound that blocks mast cells exocytosis such as the Rigel's pharmaceuticals R112.

The expression “patients exposed to chemical or biological weapons” as referred herein includes accidental or terrorist or war exposure to different chemical or biological toxic or lethal entities, comprising bacterial toxins, hydrocarbons, pesticides, heavy metal, vesicants, organochlorine agents, alkylating agents, for example sulfur mustard (2,2′-dichlorodiethyl sulfide; SM or HD) and derivatives thereof, nerve agents, blister agents and Bacillus anthracis (Anthrax).

Preferred compounds are c-kit inhibitor, more particularly a non-toxic, selective and potent c-kit inhibitor. Such inhibitors can be selected from the group consisting of 2-(3-Substitutedaryl)amino-4-aryl-thiazoles such as 2-(3-amino)arylamino-4-aryl-thiazoles, 2-aminoaryloxazoles, pyrimidine derivatives, pyrrolopyrimidine derivatives, quinazoline derivatives, quinoxaline derivatives, pyrazoles derivatives, bis monocyclic, bicyclic or heterocyclic aryl compounds, vinylene-azaindole derivatives and pyridyl-quinolones derivatives, styryl compounds, styryl-substituted pyridyl compounds, seleoindoles, selenides, tricyclic polyhydroxylic compounds and benzylphosphonic acid compounds.

Among preferred compounds, it is of interest to focus on pyrimidine derivatives such as N-phenyl-2-pyrimidine-amine derivatives (U.S. Pat. No. 5,521,184 and WO 99/03854), indolinone derivatives and pyrrol-substituted indolinones (U.S. Pat. No. 5,792,783, EP 934 931, U.S. Pat. No. 5,834,504), U.S. Pat. No. 5,883,116, U.S. Pat. No. 5,883,113, U.S. Pat. No. 5,886,020, WO 96/40116 and WO 00/38519), as well as bis monocyclic, bicyclic aryl and heteroaryl compounds (EP 584 222, U.S. Pat. No. 5,656,643 and WO 92/20642), quinazoline derivatives (EP 602 851, EP 520 722, U.S. Pat. No. 3,772,295 and U.S. Pat. No. 4,343,940), 4-amino-substituted quinazolines (U.S. Pat. No. 3,470,182), 4-thienyl-2-(1H)-quinazolones, 6,7-dialkoxyquinazolines (U.S. Pat. No. 3,800,039), aryl and heteroaryl quinazoline (U.S. Pat. No. 5,721,237, U.S. Pat. No. 5,714,493, U.S. Pat. No. 5,710,158 and WO 95/15758), 4-anilinoquinazoline compounds (U.S. Pat. No. 4,464,375), and 4-thienyl-2-(1H)-quinazolones (U.S. Pat. No. 3,551,427).

So, preferably, the invention relates to a method for treating patients exposed to chemical or biological weapons comprising administering a non toxic, potent and selective c-kit inhibitor is a pyrimidine derivatives, more particularly N-phenyl-2-pyrimidine-amine derivatives of formula I: 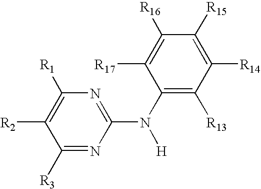
wherein the R1, R2, R3, R13 to R17 groups have the meanings depicted in EP 564 409 B1, incorporated herein in the description.

Preferably, the N-phenyl-2-pyrimidine-amine derivative is selected from the compounds corresponding to formula II: 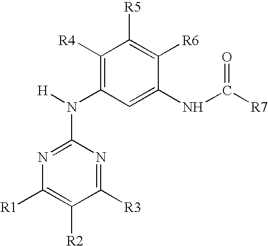
Wherein R1, R2 and R3 are independently chosen from H, F, Cl, Br, I, a C1-C5 alkyl or a cyclic or heterocyclic group, especially a pyridyl group;
R4, R5 and R6 are independently chosen from H, F, Cl, Br, I, a C1-C5 alkyl, especially a methyl group;
and R7 is a phenyl group bearing at least one substituent, which in turn possesses at least one basic site, such as an amino function.

Preferably, R7 is the following group:

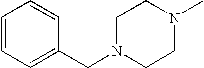

Among these compounds, the preferred are defined as follows:

R1 is a heterocyclic group, especially a pyridyl group,

R2 and R3 are H,

R4 is a C1-C3 alkyl, especially a methyl group,

R5 and R6 are H,

and R7 is a phenyl group bearing at least one substituent, which in turn possesses at least one

basic site, such as an amino function, for example the group:

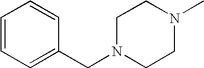

Therefore, in a preferred embodiment, the invention relates to a method for treating patients exposed to chemical or biological weapons comprising the administration of an effective amount of the compound known in the art as CGP57148B:
4-(4-méthylpipérazine-1-ylméthyl)-N-[4-méthyl-3-(4-pyridine-3-yl)pyrimidine-2 ylamino)phényl]-benzamide corresponding to the following formula:

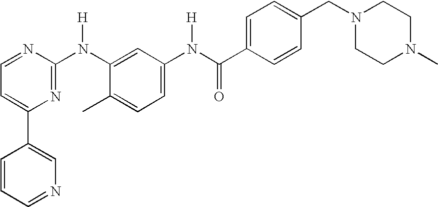

The preparation of this compound is described in example 21 of EP 564 409 and the β-form, which is particularly useful is described in WO 99/03854.

In another preferred embodiment, the invention contemplates the method mentioned above, wherein said c-kit inhibitor is selected from 2-(3-Substitutedaryl)amino-4-aryl-thiazoles such as those for which the applicant filed PCT/IB2005/000401, incorporated herein by reference, especially compounds of formula III: 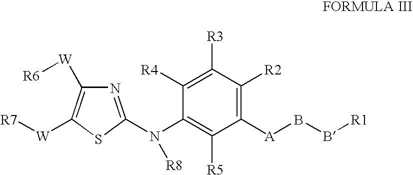
wherein

R⁶ and R⁷ are independently from each other chosen from one of the following:

i) hydrogen, a halogen (selected from F, Cl, Br or I),

ii) an alkyl¹ group defined as a linear, branched or cycloalkyl group containing from 1 to 10 carbon atoms, or from 2 or 3 to 10 carbon atoms, (for example methyl, ethyl, propyl, butyl, pentyl, hexyl . . . ) and optionally substituted with one or more hetereoatoms such as halogen (selected from F, Cl, Br or I), oxygen, and nitrogen (the latter optionally in the form of a pendant basic nitrogen functionality); as well as trifluoromethyl, carboxyl, cyano, nitro, formyl;

(iii) an aryl¹ group defined as phenyl or a substituted variant thereof bearing any combination, at any one ring position, of one or more substituents such as

halogen (selected from I, F, Cl or Br);
an alkyl¹ group;
a cycloalkyl, aryl or heteroaryl group optionally substituted by a pendant basic nitrogen functionality;
trifluoromethyl, O-alkyl¹, carboxyl, cyano, nitro, formyl, hydroxy, NH-alkyl¹, N(alkyl¹)(alkyl¹), and amino, the latter nitrogen substituents optionally in the form of a basic nitrogen functionality;
   (iv) a heteroaryl¹ group defined as a pyridyl, pyrimidinyl, pyrazinyl, pyridazinyl, thienyl, thiazolyl, imidazolyl, pyrazolyl, pyrrolyl, furanyl, oxazolyl, isoxazolyl, triazolyl, tetrazolyl, indolyl, benzimidazole, quinolinyl group, which may additionally bear any combination, at any one ring position, of one or more substituents such as
halogen (selected from F, Cl, Br or I);
an alkyl¹ group;
a cycloalkyl, aryl or heteroaryl group optionally substituted by a pendant basic nitrogen functionality,
trifluoromethyl, O-alkyl¹, carboxyl, cyano, nitro, formyl, hydroxy, NH-alkyl¹, N(alkyl¹)(alkyl¹), and amino, the latter nitrogen substituents optionally in the form of a basic nitrogen functionality;
   (v) trifluoromethyl, carboxyl, cyano, nitro, formyl, hydroxy, N(alkyl¹)(alkyl¹), and amino, the latter nitrogen substituents optionally in the form of a basic nitrogen functionality.

R⁸ is one of the following:

(i) hydrogen, or

(ii) a linear or branched alkyl group containing from 1 to 10 carbon atoms and optionally substituted with one or more hetereoatoms such as halogen (selected from F, Cl, Br or I), oxygen, and nitrogen, the latter optionally in the form of a pendant basic nitrogen functionality, or

(iii) CO—R8 or COOR8 or CONHR8 or SO2R8 wherein R8 may be

a linear or branched alkyl group containing from 1 to 10 carbon atoms and optionally substituted with one or more hetereoatoms such as halogen (selected from F, Cl, Br or I), oxygen, and nitrogen, the latter optionally in the form of a pendant basic nitrogen functionality, or
an aryl group such as phenyl or a substituted variant thereof bearing any combination, at any one ring position, of one or more substituents such as halogen (selected from F, Cl, Br or I), alkyl groups containing from 1 to 10 carbon atoms and optionally substituted with one or more hetereoatoms such as halogen (selected from F, Cl, Br or I), oxygen, and nitrogen, the latter optionally in the form of a pendant basic nitrogen functionality; as well as trifluoromethyl, C₁₋₆alkyloxy, carboxyl, cyano, nitro, formyl, hydroxy, C₁₋₆alkylamino, di(C₁₋₆alkyl)amino, and amino, the latter nitrogen substituents optionally in the form of a pendant basic nitrogen functionality; as well as CO—R, COO—R, CONH—R, SO2-R, and SO2NH—R wherein R is a linear or branched alkyl group containing from 1 to 10 carbon atoms and optionally substituted with at least one heteroatom, notably a halogen (selected from F, Cl, Br or I), oxygen, and nitrogen, the latter optionally in the form of a pendant basic nitrogen functionality, or
a heteroaryl group such as a pyridyl, pyrimidinyl, pyrazinyl, pyridazinyl, thienyl, thiazolyl, imidazolyl, pyrazolyl, pyrrolyl, furanyl, oxazolyl, isoxazolyl, triazolyl, tetrazolyl, indolyl, benzimidazole, quinolinyl group, which may additionally bear any combination, at any one ring position, of one or more substituents such as halogen (selected from F, Cl, Br or I), alkyl groups containing from 1 to 10 carbon atoms and optionally substituted with one or more hetereoatoms such as halogen (selected from F, Cl, Br or I), oxygen, and nitrogen, the latter optionally in the form of a pendant basic nitrogen functionality; as well as trifluoromethyl, C₁₋₆alkyloxy, carboxyl, cyano, nitro, formyl, hydroxy, C₁₋₆alkylamino, di(C₁₋₆alkyl)amino, and amino, the latter nitrogen substituents optionally in the form of a basic nitrogen functionality; as well as CO—R, COO—R, CONH—R, SO2-R, and SO2NH—R wherein R is a linear or branched alkyl group containing from 1 to 10 carbon atoms and optionally substituted with at least one heteroatom, notably a halogen (selected from F, Cl, Br or I), oxygen, and nitrogen, the latter optionally in the form of a pendant basic nitrogen functionality.

R2, R3, R4 and R5 each independently are selected from hydrogen, halogen (selected from F, Cl, Br or I), a linear or branched alkyl group containing from 1 to 10 carbon atoms and optionally substituted with one or more hetereoatoms such as halogen (selected from F, Cl, Br or I), oxygen, and nitrogen, the latter optionally in the form of a pendant basic nitrogen functionality; as well as trifluoromethyl, C₁₋₆alkyloxy, amino, C₁₋₆alkylamino, di(C₁₋₆alkyl)amino, carboxyl, cyano, nitro, formyl, hydroxy, and CO—R, COO—R, CONH—R, SO2-R, and SO2NH—R wherein R is a linear or branched alkyl group containing from 1 to 10 carbon atoms and optionally substituted with at least one heteroatom, notably a halogen (selected from F, Cl, Br or I), oxygen, and nitrogen, the latter optionally in the form of a pendant basic nitrogen functionality.

A is: CH2, O, S, SO2, CO, or COO,

B is a bond or NH, NCH3, NR*, (CH2)n (n is 0, 1 or 2), O, S, SO2, CO, or COO,

B′ is a bond or NH, NCH3, NR*, (CH2)n (n is 0, 1 or 2), O, S, SO2, CO or COO;

R* being an alkyl¹, aryl¹ or heteroaryl¹

W is a bond or a linker selected from NH, NHCO, NHCOO, NHCONH, NHSO2, NHSO2NH, CO, CONH, COO, COCH2, (CH2)n (n is 0, 1 or 2), CH2-CO, CH2COO, CH2-NH, O, OCH2, S, SO2, and SO2NH

R¹ is:

a) a linear or branched alkyl group containing from 1 to 10 carbon atoms optionally substituted with at least one heteroatom, notably a halogen selected from I, Cl, Br and F, and/or bearing a pendant basic nitrogen functionality;

b) an aryl or heteroaryl group optionally substituted by an alkyl or aryl group optionally substituted with a heteroatom, notably a halogen selected from I, Cl, Br and F or bearing a pendant basic nitrogen functionality

c) an alkyl¹, aryl¹ or heteroaryl¹.

It will be understood that a C1-C10 alkyl encompasses a methyl, ethyl, propyl, and a C2 to C4 alkyl or a C2 to C10 alkyl.

For example, a subset of compounds may correspond to

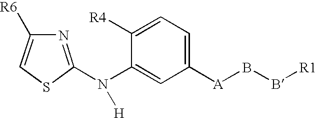

Wherein R1, R4 and R6 have the meaning as defined above.

It will be understood that A-B—B′ includes but is not limited to:

CH2, CH2-CO, CH2-CO—CH2, CH2COO, CH2-CH2-CO, CH2-CH2-COO, CH2-NH, CH2-CH2-NH, CH2-NH—CH2 or CH2-NH—CO or CH2-CO—NH

It will be understood that A-B—B′ also includes but is not limited to:

CO—CH2, COO—CH2, CO—CH2-CH2, CO—NH, or CO—NH—CH2

as well as O—CH2

It will also be understood that NH in B or B′ can also be NCH3

In the above formula III, when W is other than a single bond, it will be understood that A can be also be NH or NCH3.

In the above formula, the following combinations are contemplated:
R6 is (iv), R4 is H or CH3, A-B—B′ is CO—NH and R1 is as defined above.
R6 is (iv), R4 is H or CH3, A-B—B′ is CH2-CO—NH and R1 is as defined above.
R6 is (iv), R4 is H or CH3, A-B—B′ is CH2-CO and R1 is as defined above.
R6 is (iv), R4 is H or CH3, A-B—B′ is CH2-NH—CO and R1 is as defined above.
R6 is (iv), R4 is H or CH3, A-B—B′ is CH2-NH and R1 is as defined above.
R6 is (iv), R4 is H or CH3, A-B—B′ is CH2 and R1 is as defined above.
R6 is W-(iv), R4 is a C₁-C₂ alkyl, A-B—B′ is CO—NH and R1 is as defined above.
R6 is (iv), R4 is a C1-C2 alkyl, A-B—B′ is CH2-CO—NH and R1 is as defined above.
R6 is (iv), R4 is a C1-C2 alkyl, A-B—B′ is CH2-CO and R1 is as defined above.
R6 is a pyridyl according to (iv), R4 is a C1-C2 alkyl, A-B—B′ is CO—NH, CH2-CO—NH, CH2-CO, CH2-NH, CH2-NH—CO and R1 is as defined above.

In the above combination, R1 can be an alkyl¹.

In the above combination, R1 can be an aryl¹.

In the above combination, R1 can be an heteroaryl¹.

In another preferred embodiment, the invention contemplated the method mentioned above, wherein said c-kit inhibitor is selected from 2-(3-amino)arylamino-4-aryl-thiazoles such as those for which the applicant filed WO 2004/014903, incorporated herein in the description, especially compounds of formula IV: 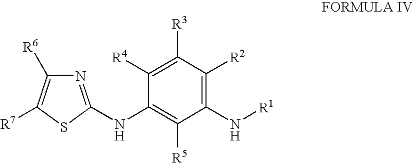
and wherein R¹ is:
a) a linear or branched alkyl group containing from 1 to 10 carbon atoms optionally substituted with at least one heteroatom, notably a halogen selected from I, Cl, Br and F, and/or bearing a pendant basic nitrogen functionality;
b) an aryl or heteroaryl group optionally substituted by an alkyl or aryl group optionally substituted with a heteroatom, notably a halogen selected from I, Cl, Br and F or bearing a pendant basic nitrogen functionality;
c) a —CO—NH—R, —CO—R, —CO—OR or a —CO—NRR′ group, wherein R and R′ are independently chosen from H or an aryl, heteroaryl, alkyl and cycloalkyl group optionally substituted with at least one heteroatom, notably a halogen selected from I, Cl, Br and F, and/or bearing a pendant basic nitrogen functionality;
R² is hydrogen, halogen or a linear or branched alkyl group containing from 1 to 10 carbon atoms, trifluoromethyl or alkoxy;
R³ is hydrogen, halogen or a linear or branched alkyl group containing from 1 to 10 carbon atoms, trifluoromethyl or alkoxy;
R⁴ is hydrogen, halogen or a linear or branched alkyl group containing from 1 to 10 carbon atoms, trifluoromethyl or alkoxy;
R⁵ is hydrogen, halogen or a linear or branched alkyl group containing from 1 to 10 carbon atoms, trifluoromethyl or alkoxy;
R⁶ is one of the following:
(i) an aryl group such as phenyl or a substituted variant thereof bearing any combination, at any one ring position, of one or more substituents such as halogen, alkyl groups containing from 1 to 10 carbon atoms, trifluoromethyl, and alkoxy;
(ii) a heteroaryl group such as a 2, 3, or 4-pyridyl group, which may additionally bear any combination of one or more substituents such as halogen, alkyl groups containing from 1 to 10 carbon atoms, trifluoromethyl and alkoxy;
(iii) a five-membered ring aromatic heterocyclic group such as for example 2-thienyl, 3-thienyl, 2-thiazolyl, 4-thiazolyl, 5-thiazolyl, which may additionally bear any combination of one or more substituents such as halogen, an alkyl group containing from 1 to 10 carbon atoms, trifluoromethyl, and alkoxy,
iv) H, a halogen selected from I, F, Cl or Br; NH2, NO2 or SO2-R, wherein R is a linear or branched alkyl group containing one or more group such as 1 to 10 carbon atoms, and optionally substituted with at least one heteroatom, notably a halogen selected from I, Cl, Br and F, and/or bearing a pendant basic nitrogen functionality;
and R⁷ is one of the following:
(i) an aryl group such as phenyl or a substituted variant thereof bearing any combination, at any one ring position, of one or more substituents such as halogen, alkyl groups containing from 1 to 10 carbon atoms, trifluoromethyl, and alkoxy;
(ii) a heteroaryl group such as a 2, 3, or 4-pyridyl group, which may additionally bear any combination of one or more substituents such as halogen, alkyl groups containing from 1 to 10 carbon atoms, trifluoromethyl and alkoxy;
(iii) a five-membered ring aromatic heterocyclic group such as for example 2-thienyl, 3-thienyl, 2-thiazolyl, 4-thiazolyl, 5-thiazolyl, which may additionally bear any combination of one or more substituents such as halogen, an alkyl group containing from 1 to 10 carbon atoms, trifluoromethyl, and alkoxy.
iv) H, a halogen selected from I, F, Cl or Br; NH2, NO2 or SO2-R, wherein R is a linear or branched alkyl group containing one or more group such as 1 to 10 carbon atoms, and optionally substituted with at least one heteroatom, notably a halogen selected from I, Cl, Br and F, and/or bearing a pendant basic nitrogen functionality.

In another preferred embodiment, when R¹ has the meaning depicted in c) above, the invention is directed to compounds of the following formulas: 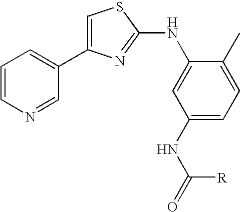
wherein R is H or an organic group that can be selected for example from a linear or branched alkyl group containing from 1 to 10 carbon atoms optionally substituted with at least one heteroatom or bearing a pendant basic nitrogen functionality; a cycloalkyl, an aryl or heteroaryl group optionally substituted by an alkyl, a cycloalkyl, an aryl or heteroaryl group optionally substituted with a heteroatom, notably a halogen selected from I, Cl, Br and F and/or bearing a pendant basic nitrogen functionality.

Among the particular compounds in which R1 has the meaning as depicted in c) above, the invention is directed to amide-aniline, amide-benzylamine, amide-phenol, urea compounds of the following formulas respectively: 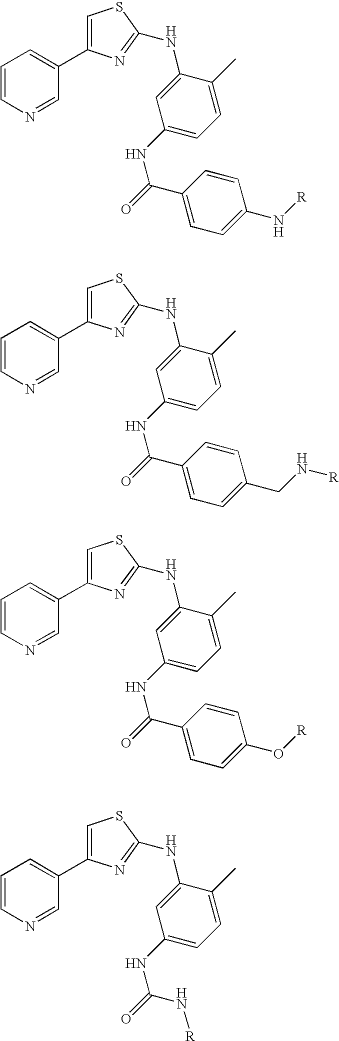
wherein R is H or an organic group that can be selected for example from a linear or branched alkyl group containing from 1 to 10 carbon atoms optionally substituted with at least one heteroatom or bearing a pendant basic nitrogen functionality; a cycloalkyl, an aryl or heteroaryl group optionally substituted with a heteroatom, notably a halogen selected from I, Cl, Br and F and/or bearing a pendant basic nitrogen functionality; or a a cycloalkyl, an aryl or heteroaryl group optionally substituted with a cycloalkyl, an aryl or heteroaryl group optionally substituted with a heteroatom, notably a halogen selected from I, Cl, Br and F and/or bearing a pendant basic nitrogen functionality;
a —SO2-R group wherein R is an alkyl, cycloalkyl, aryl or heteroaryl optionally substituted with an heteroatom, notably a halogen selected from I, Cl, Br and F and/or bearing a pendant basic nitrogen functionality; or a —CO—R or a —CO—NRR′ group, wherein R and R′ are independently chosen from H, an alkyl, a cycloalkyl, an aryl or heteroaryl group optionally substituted with at least one heteroatom, notably selected from I, Cl, Br and F, and/or bearing a pendant basic nitrogen functionality.

Among the particular compounds in which R1 has the meaning as depicted in a) and b) above, the invention is directed to N-Aminoalkyl-N′-thiazol-2-yl-benzene-1,3-diamine compounds of the following formula IVbis: 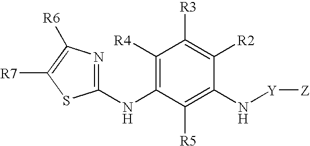
wherein Y is a linear or branched alkyl group containing from 1 to 10 carbon atoms;
wherein Z represents an aryl or heteroaryl group, optionally substituted at one or more ring position with any permutation of the following groups:
a halogen such as F, Cl, Br, I;
a linear or branched alkyl group containing from 1 to 10 carbon atoms optionally substituted with at least one heteroatom (for example a halogen) and/or bearing a pendant basic nitrogen functionality; a cycloalkyl, an aryl or heteroaryl group optionally substituted with at least one heteroatom, notably a halogen selected from I, Cl, Br and F, and/or bearing a pendant basic nitrogen functionality; or a cycloalkyl, an aryl or heteroaryl group substituted by an alkyl, a cycloalkyl, an aryl or heteroaryl group optionally substituted with an heteroatom, notably a halogen selected from I, Cl, Br and F, and/or bearing a pendant basic nitrogen functionality;
an O—R, where R is a linear or branched alkyl group containing from 1 to 10 carbon atoms optionally substituted with at least one heteroatom (for example a halogen) and/or bearing a pendant basic nitrogen functionality; a cycloalkyl, an aryl or heteroaryl group optionally substituted with at least one heteroatom, notably a halogen selected from I, Cl, Br and F, and/or bearing a pendant basic nitrogen functionality; or a cycloalkyl, an aryl or heteroaryl group substituted by an alkyl, a cycloalkyl, an aryl or heteroaryl group optionally substituted with an heteroatom, notably a halogen selected from I, Cl, Br and F, and/or bearing a pendant basic nitrogen functionality;
an NRaRb, where Ra and Rb represents a hydrogen, or a linear or branched alkyl group containing from 1 to 10 carbon atoms optionally substituted with at least one heteroatom (for example a halogen) and/or bearing a pendant basic nitrogen functionality or a cycle; a cycloalkyl, an aryl or heteroaryl group optionally substituted with at least one heteroatom, notably a halogen selected from I, Cl, Br and F, and/or bearing a pendant basic nitrogen functionality; or a cycloalkyl, an aryl or heteroaryl group substituted by an alkyl, a cycloalkyl, an aryl or heteroaryl group optionally substituted with an heteroatom, notably a halogen selected from I, Cl, Br and F, and/or bearing a pendant basic nitrogen functionality;
a COOR, where R is a linear or branched alkyl group containing from 1 to 10 carbon atoms optionally substituted with at least one heteroatom (for example a halogen) and/or bearing a pendant basic nitrogen functionality; a cycloalkyl, an aryl or heteroaryl group optionally substituted with at least one heteroatom, notably a halogen selected from I, Cl, Br and F, and/or bearing a pendant basic nitrogen functionality; or a cycloalkyl, an aryl or heteroaryl group substituted by an alkyl, a cycloalkyl, an aryl or heteroaryl group optionally substituted with an heteroatom, notably a halogen selected from I, Cl, Br and F, and/or bearing a pendant basic nitrogen functionality;
a CONRaRb, where Ra and Rb are a hydrogen or a linear or branched alkyl group containing from 1 to 10 carbon atoms optionally substituted with at least one heteroatom (for example a halogen) and/or bearing a pendant basic nitrogen functionality; a cycloalkyl, an aryl or heteroaryl group optionally substituted with at least one heteroatom, notably a halogen selected from I, Cl, Br and F, and/or bearing a pendant basic nitrogen functionality; or a cycloalkyl, an aryl or heteroaryl group substituted by an alkyl, a cycloalkyl, an aryl or heteroaryl group optionally substituted with an heteroatom, notably a halogen selected from I, Cl, Br and F, and/or bearing a pendant basic nitrogen functionality;
an NHCOR, where R is a linear or branched alkyl group containing from 1 to 10 carbon atoms optionally substituted with at least one heteroatom (for example a halogen) and/or bearing a pendant basic nitrogen functionality; a cycloalkyl, an aryl or heteroaryl group optionally substituted with at least one heteroatom, notably a halogen selected from I, Cl, Br and F, and/or bearing a pendant basic nitrogen functionality; or a cycloalkyl, an aryl or heteroaryl group substituted by an alkyl, a cycloalkyl, an aryl or heteroaryl group optionally substituted with an heteroatom, notably a halogen selected from I, Cl, Br and F, and/or bearing a pendant basic nitrogen functionality;
an NHCOOR, where R is a linear or branched alkyl group containing from 1 to 10 carbon atoms optionally substituted with at least one heteroatom (for example a halogen) and/or bearing a pendant basic nitrogen functionality; a cycloalkyl, an aryl or heteroaryl group optionally substituted with at least one heteroatom, notably a halogen selected from I, Cl, Br and F, and/or bearing a pendant basic nitrogen functionality; or a cycloalkyl, an aryl or heteroaryl group substituted by an alkyl, a cycloalkyl, an aryl or heteroaryl group optionally substituted with an heteroatom, notably a halogen selected from I, Cl, Br and F, and/or bearing a pendant basic nitrogen functionality;
an NHCONRaRb, where Ra and Rb are a hydrogen or a linear or branched alkyl group containing from 1 to 10 carbon atoms optionally substituted with at least one heteroatom (for example a halogen) and/or bearing a pendant basic nitrogen functionality; a cycloalkyl, an aryl or heteroaryl group optionally substituted with at least one heteroatom, notably a halogen selected from I, Cl, Br and F, and/or bearing a pendant basic nitrogen functionality; or a cycloalkyl, an aryl or heteroaryl group substituted by an alkyl, a cycloalkyl, an aryl or heteroaryl group optionally substituted with an heteroatom, notably a halogen selected from I, Cl, Br and F, and/or bearing a pendant basic nitrogen functionality;
an OSO₂R, where R is a linear or branched alkyl group containing from 1 to 10 carbon atoms optionally substituted with at least one heteroatom (for example a halogen) and/or bearing a pendant basic nitrogen functionality; a cycloalkyl, an aryl or heteroaryl group optionally substituted with at least one heteroatom, notably a halogen selected from I, Cl, Br and F, and/or bearing a pendant basic nitrogen functionality; or a cycloalkyl, an aryl or heteroaryl group substituted by an alkyl, a cycloalkyl, an aryl or heteroaryl group optionally substituted with an heteroatom, notably a halogen selected from I, Cl, Br and F, and/or bearing a pendant basic nitrogen functionality;
an NRaOSO₂Rb, where Ra and Rb are a linear or branched alkyl group containing from 1 to 10 carbon atoms optionally substituted with at least one heteroatom (for example a halogen) and/or bearing a pendant basic nitrogen functionality; Ra can also be a hydrogen; a cycloalkyl, an aryl or heteroaryl group optionally substituted with at least one heteroatom, notably a halogen selected from I, Cl, Br and F, and/or bearing a pendant basic nitrogen functionality; or a cycloalkyl, an aryl or heteroaryl group substituted by an alkyl, a cycloalkyl, an aryl or heteroaryl group optionally substituted with an heteroatom, notably a halogen selected from I, Cl, Br and F, and/or bearing a pendant basic nitrogen functionality;
   R² is hydrogen, halogen or a linear or branched alkyl group containing from 1 to 10 carbon atoms, trifluoromethyl or alkoxy;
   R³ is hydrogen, halogen or a linear or branched alkyl group containing from 1 to 10 carbon atoms, trifluoromethyl or alkoxy;
   R⁴ is hydrogen, halogen or a linear or branched alkyl group containing from 1 to 10 carbon atoms, trifluoromethyl or alkoxy;
   R⁵ is hydrogen, halogen or a linear or branched alkyl group containing from 1 to 10 carbon atoms, trifluoromethyl or alkoxy;
   R⁶ is one of the following:
   (i) an aryl group such as phenyl or a substituted variant thereof bearing any combination, at any one ring position, of one or more substituents such as halogen, alkyl groups containing from 1 to 10 carbon atoms, trifluoromethyl, and alkoxy;
   (ii) a heteroaryl group such as a 2, 3, or 4-pyridyl group, which may additionally bear any combination of one or more substituents such as halogen, alkyl groups containing from 1 to 10 carbon atoms, trifluoromethyl and alkoxy;
   (iii) a five-membered ring aromatic heterocyclic group such as for example 2-thienyl, 3-thienyl, 2-thiazolyl, 4-thiazolyl, 5-thiazolyl, which may additionally bear any combination of one or more substituents such as halogen, an alkyl group containing from 1 to 10 carbon atoms, trifluoromethyl, and alkoxy.
   iv) H, a halogen selected from I, F, Cl or Br; NH2, NO2 or SO2-R, wherein R is a linear or branched alkyl group containing one or more group such as 1 to 10 carbon atoms, and optionally substituted with at least one heteroatom, notably a halogen selected from I, Cl, Br and F, and/or bearing a pendant basic nitrogen functionality;
   and R⁷ is one of the following:
   (i) an aryl group such as phenyl or a substituted variant thereof bearing any combination, at any one ring position, of one or more substituents such as halogen, alkyl groups containing from 1 to 10 carbon atoms, trifluoromethyl, and alkoxy;
   (ii) a heteroaryl group such as a 2, 3, or 4-pyridyl group, which may additionally bear any combination of one or more substituents such as halogen, alkyl groups containing from 1 to 10 carbon atoms, trifluoromethyl and alkoxy;
   (iii) a five-membered ring aromatic heterocyclic group such as for example 2-thienyl, 3-thienyl, 2-thiazolyl, 4-thiazolyl, 5-thiazolyl, which may additionally bear any combination of one or more substituents such as halogen, an alkyl group containing from 1 to 10 carbon atoms, trifluoromethyl, and alkoxy.
   iv) H, an halogen selected from I, F, Cl or Br; NH2, NO2 or SO2-R, wherein R is a linear or branched alkyl group containing one or more group such as 1 to 10 carbon atoms, and optionally substituted with at least one heteroatom, notably a halogen selected from I, Cl, Br and F, and/or bearing a pendant basic nitrogen functionality.

It will be understood that a C1-C10 alkyl encompasses a methyl, ethyl, propyl, and a C2 to C4 alkyl or a C2 to C10 alkyl.

An example of preferred compounds of the above formula is depicted below:
4-{[4-Methyl-3-(4-pyridin-3-yl-thiazol-2-ylamino)-phenylamino]-methyl}-benzoic acid methyl ester

Among the compounds of formula III or IV, the invention is particularly embodied by the compounds of the following formula V: 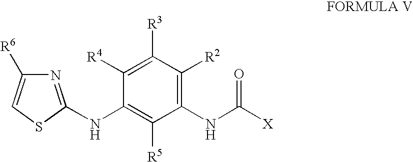
wherein X is R or NRR′ and wherein R and R′ are independently chosen from H, an aryl, a heteroaryl, an alkyl, or a cycloalkyl group optionally substituted with at least one heteroatom, such as for example a halogen chosen from F, I, Cl and Br and optionally bearing a pendant basic nitrogen functionality; or an aryl, a heteroaryl, an alkyl or a cycloalkyl group substituted with an aryl, a heteroaryl, an alkyl or a cycloalkyl group optionally substituted with at least one heteroatom, such as for example a halogen chosen from F, I, Cl and Br and optionally bearing a pendant basic nitrogen functionality,
R² is hydrogen, halogen or a linear or branched alkyl group containing from 1 to 10 carbon atoms, trifluoromethyl or alkoxy;
R³ is hydrogen, halogen or a linear or branched alkyl group containing from 1 to 10 carbon atoms, trifluoromethyl or alkoxy;
R⁴ is hydrogen, halogen or a linear or branched alkyl group containing from 1 to 10 carbon atoms, trifluoromethyl or alkoxy;
R⁵ is hydrogen, halogen or a linear or branched alkyl group containing from 1 to 10 carbon atoms, trifluoromethyl or alkoxy;
R⁶ is one of the following:
(i) an aryl group such as phenyl or a substituted variant thereof bearing any combination, at any one ring position, of one or more substituents such as halogen, alkyl groups containing from 1 to 10 carbon atoms, trifluoromethyl, and alkoxy;
(ii) a heteroaryl group such as a 2, 3, or 4-pyridyl group, which may additionally bear any combination of one or more substituents such as halogen, alkyl groups containing from 1 to 10 carbon atoms, trifluoromethyl and alkoxy;
(iii) a five-membered ring aromatic heterocyclic group such as for example 2-thienyl, 3-thienyl, 2-thiazolyl, 4-thiazolyl, 5-thiazolyl, which may additionally bear any combination of one or more substituents such as halogen, an alkyl group containing from 1 to 10 carbon atoms, trifluoromethyl, and alkoxy.
iv) H, a halogen selected from I, F, Cl or Br; NH2, NO2 or SO2-R, wherein R is a linear or branched alkyl group containing one or more group such as 1 to 10 carbon atoms, and optionally substituted with at least one heteroatom, notably a halogen selected from I, Cl, Br and F, and/or bearing a pendant basic nitrogen functionality.

In another alternative, substituent R6, which in the formula II is connected to position 4 of the thiazole ring, may instead occupy position 5 of the thiazole ring.

Among the preferred compounds corresponding formula III, IV or V, the invention is directed to compounds in which R1 or X is a substituted alkyl, aryl or heteroaryl group bearing a pendant basic nitrogen functionality represented for example by the structures a to f and g to m shown below, wherein the wavy line corresponds to the point of attachment to core structure of formula III, IV or V:

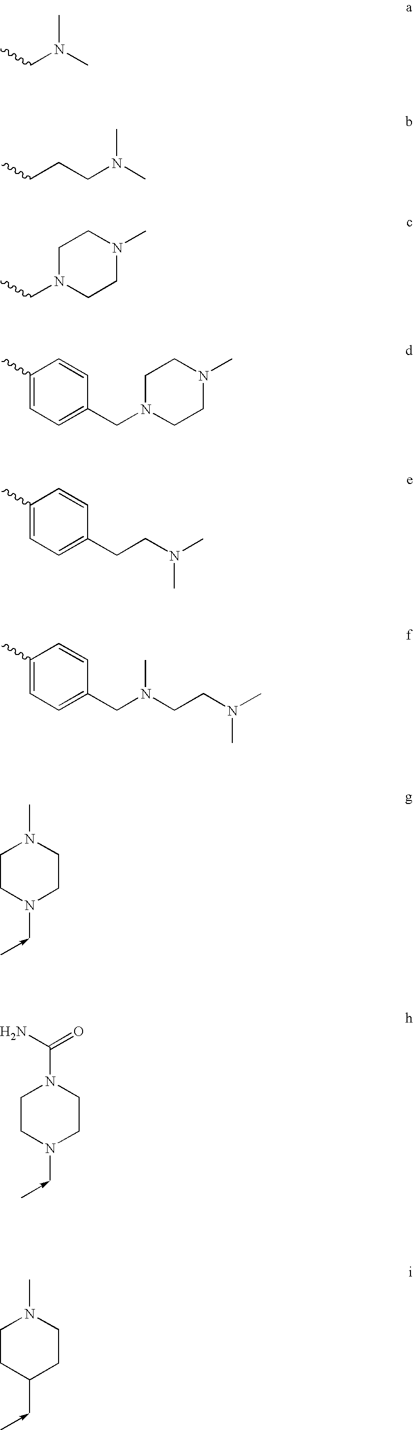

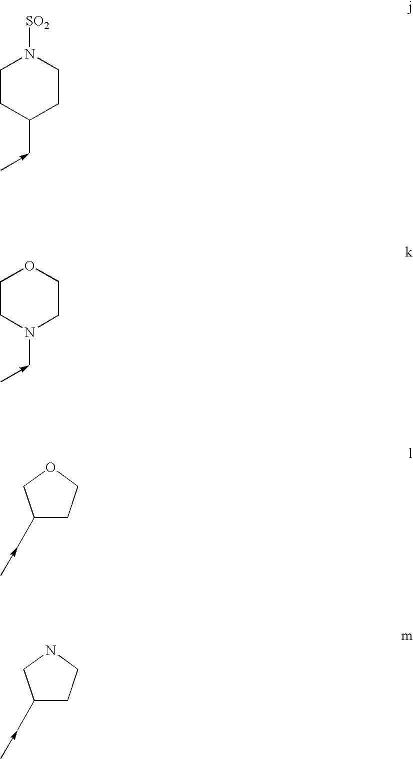

Among group a to f, is preferentially group d. Also, for g to m, the arrow may include a point of attachment to the core structure via a phenyl group.

Furthermore, among the preferred compounds of formula III, IV or V, the invention concerns the compounds in which R² and R³ are hydrogen. Preferentially, R⁴ is a methyl group and R⁵ is H. In addition, R⁶ is preferentially a 3-pyridyl group (cf. structure g below), or a 4-pyridyl group (cf. structure h below) or a benzonitrile group. The wavy line in structure g and h correspond to the point of attachment to the core structure of formula III, IV or V.

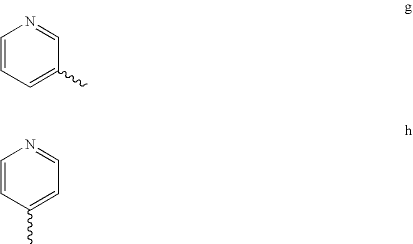

Alternatively, among the preferred compounds of formula III, IV or V, the invention concerns the compounds in which R6 or R7 is preferentially a cyanophenyl group as shown below, wherein the wavy line in structure p and q correspond to the point of attachment to the core structure of formula III, IV or V:

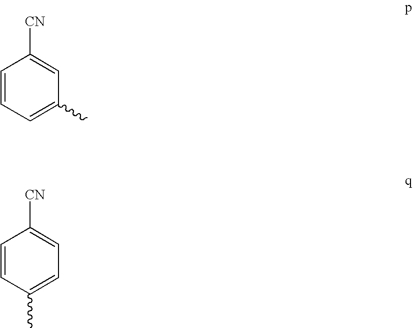

In one particular embodiment, R1 in formula III and IV, X in formula V and Z in formula IVbis can be: 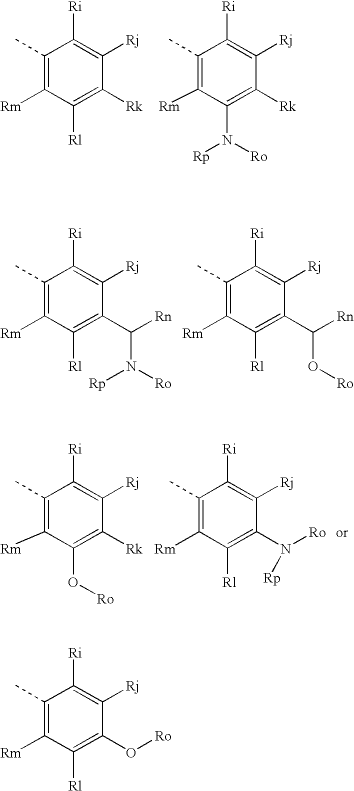
wherein Ri, Rj, Rk, Rl, Rm, Ro, and Rp are independently chosen from —H, an halogen such as Cl, F, Br, I; a trifluoromethyl group, a CN group, SO2, OH, or a group selected for example from a linear or branched alkyl group containing from 1 to 10 carbon atoms optionally substituted with at least one heteroatom and/or bearing a pendant basic nitrogen functionality; a cycloalkyl, an aryl or heteroaryl group optionally substituted with a heteroatom, notably a halogen selected from I, Cl, Br and F or bearing a pendant basic nitrogen functionality; or a cycloalkyl, an aryl or heteroaryl group optionally substituted with a cycloalkyl, an aryl or heteroaryl group optionally substituted with an heteroatom, notably a halogen selected from I, Cl, Br and F or bearing a pendant basic nitrogen functionality;
a NRR′, NRCOR, NRCONR′R″, NROSO2R′, SO2-R, COOR, CONRR′, NHCOOR, CO—R, CO—NRR′, OR or OSO₂R group where R and R′ are independently chosen from H or a linear or branched alkyl group containing from 1 to 10 carbon atoms optionally substituted with at least one heteroatom and/or bearing a pendant basic nitrogen functionality; a cycloalkyl, an aryl or heteroaryl group optionally substituted with a heteroatom, notably a halogen selected from I, Cl, Br and F or bearing a pendant basic nitrogen functionality; or a cycloalkyl, an aryl or heteroaryl group optionally substituted with a cycloalkyl, an aryl or heteroaryl group optionally substituted with an heteroatom, notably a halogen selected from I, Cl, Br and F or bearing a pendant basic nitrogen functionality.

For example, one of Ri, Rj, Rk, Rl, Rm, Ro or Rp is selected from group a, b, c, g, h, i, j, k, l, m as defined above such as Rk is one of a, b, c, g, h, i, j, k, l, m and Ri, Rj, Rl, Rm is H.

Thus, the invention contemplates:
1—A compound of formula V as depicted above, wherein X is group d and R⁶ is a 3-pyridyl group.
2—A compound of formula V as depicted above, wherein X is group d and R⁴ is a methyl group.
3—A compound of formula III or IV as depicted above, wherein R¹ is group d and R² and/or R³ and/or R⁵ is H.
4—A compound of formula III or IV as depicted above, wherein R⁶ is a 3-pyridyl group and R⁴ is a methyl group.
5—A compound of formula III or IV as depicted above, wherein R² and/or R³ and/or R⁵ is H and R⁴ is a methyl group.
6—A compound of formula III or IV as depicted above wherein R² and/or R³ and/or R⁵ is H, R⁴ is a methyl group and R⁶ is a 3-pyridyl group.

Among the compounds of formula IV, the invention is particularly embodied by the compounds wherein R2, R3, R5 are hydrogen, corresponding to the following formula 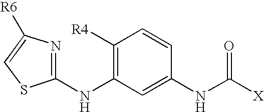
wherein X is R or NRR′ and wherein R and R′ are independently chosen from H or an organic group that can be selected for example from a linear or branched alkyl group containing from 1 to 10 carbon atoms optionally substituted with at least one heteroatom or bearing a pendant basic nitrogen functionality; a cycloalkyl, an aryl or heteroaryl group optionally substituted with an heteroatom, notably a halogen selected from I, Cl, Br and F or bearing a pendant basic nitrogen functionality; or a cycloalkyl, an aryl or heteroaryl group optionally substituted with a cycloalkyl, an aryl or heteroaryl group optionally substituted with an heteroatom, notably a halogen selected from I, Cl, Br and F or bearing a pendant basic nitrogen functionality;
a —SO2-R group wherein R is an alkyl, cycloalkyl, aryl or heteroaryl optionally substituted with a heteroatom, notably a halogen selected from I, Cl, Br and F or bearing a pendant basic nitrogen functionality; or a —CO—R or a —CO—NRR′ group, wherein R and R′ are independently chosen from H, an alkyl, a cycloalkyl, an aryl or heteroaryl group optionally substituted with at least one heteroatom, notably selected from I, Cl, Br and F, and/or bearing a pendant basic nitrogen functionality.

R⁴ is hydrogen, halogen or a linear or branched alkyl group containing from 1 to 10 carbon atoms, trifluoromethyl or alkoxy;

R⁶ is one of the following:

(i) an aryl group such as phenyl or a substituted variant thereof bearing any combination, at any one ring position, of one or more substituents such as halogen, alkyl groups containing from 1 to 10 carbon atoms, trifluoromethyl, and alkoxy;

(ii) a heteroaryl group such as a 2, 3, or 4-pyridyl group, which may additionally bear any combination of one or more substituents such as halogen, alkyl groups containing from 1 to 10 carbon atoms, trifluoromethyl and alkoxy;

(iii) a five-membered ring aromatic heterocyclic group such as for example 2-thienyl, 3-thienyl, 2-thiazolyl, 4-thiazolyl, 5-thiazolyl, which may additionally bear any combination of one or more substituents such as halogen, an alkyl group containing from 1 to 10 carbon atoms, trifluoromethyl, and alkoxy.

iv) H, a halogen selected from I, F, Cl or Br; NH2, NO2 or SO2-R, wherein R is a linear or branched alkyl group containing one or more group such as 1 to 10 carbon atoms, and optionally substituted with at least one heteroatom, notably a halogen selected from I, Cl, Br and F, and/or bearing a pendant basic nitrogen functionality.

In another alternative, substituent R6, which in the formula III is connected to position 4 of the thiazole ring, may instead occupy position 5 of the thiazole ring.

### EXAMPLES

2-(2-methyl-5-amino)phenyl-4-(3-pyridyl)-thiazole
4-(4-Methyl-piperazin-1-ylmethyl)-N-[3-(4-pyridin-3-yl-thiazol-2-ylamino)-phenyl]-benzamide
N-[4-Methyl-3-(4-phenyl-thiazol-2-ylamino)-phenyl]-4-(4-methyl-piperazin-1-ylmethyl)-benzamide
N-[3-([2,4′]Bithiazolyl-2′-ylamino)-4-methyl-phenyl]-4-(4-methyl-piperazin-1-ylmethyl)-benzamide
4-(4-Methyl-piperazin-1-ylmethyl)-N-[4-methyl-3-(4-pyrazin-2-yl-thiazol-2-ylamino)-phenyl]-benzamide
2-[5-(3-Iodo-benzoylamino)-2-methyl-phenylamino]-thiazole-4-carboxylic acid ethyl ester
2-{2-Methyl-5-[4-(4-methyl-piperazin-1-ylmethyl)-benzoylamino]-phenylamino}-thiazole-4-carboxylic acid ethyl ester
2-(2-chloro-5-amino)phenyl-4-(3-pyridyl)-thiazole
3-Bromo-N-{3-[4-(4-chloro-phenyl)-5-methyl-thiazol-2-ylamino]-4-methyl-phenyl}-benzamide
{3-[4-(4-Chloro-phenyl)-5-methyl-thiazol-2-ylamino]-4-methyl-phenyl}-carbamic acid isobutyl ester
2-[5-(3-Bromo-benzoylamino)-2-methyl-phenylamino]-5-(4-chloro-phenyl)-thiazole-4-carboxylic acid ethyl ester
2-[5-(3-Bromo-benzoylamino)-2-methyl-phenylamino]-5-(4-chloro-phenyl)-thiazole-4-carboxylic acid (2-dimethylamino-ethyl)-amide
N-{3-[4-(4-Methoxy-phenyl)-thiazol-2-ylamino]-4-methyl-phenyl}-4-(4-methyl-piperazin-1 ylmethyl)-benzamide
4-(4-Methyl-piperazin-1-ylmethyl)-N-{4-methyl-3-[4-(3-trifluoromethyl-phenyl)-thiazol-2-ylamino]-phenyl}-benzamide
N-{4-Methyl-3-[4-(3-nitro-phenyl)-thiazol-2-ylamino]-phenyl}-4-(4-methyl-piperazin-1-ylmethyl)-benzamide
N-{3-[4-(2,5-Dimethyl-phenyl)-thiazol-2-ylamino]-4-methyl-phenyl}-4-(4-methyl-piperazin-1-ylmethyl)-benzamide
N-{3-[4-(4-Chloro-phenyl)-thiazol-2-ylamino]-4-methyl-phenyl}-4-(4-methyl-piperazin-1-ylmethyl)-benzamide
N-{3-[4-(3-Methoxy-phenyl)-thiazol-2-ylamino]-4-methyl-phenyl}-4-(4-methyl-piperazin-1-ylmethyl)-benzamide
N-[4-Methyl-3-(4-pyridin-3-yl-thiazol-2-ylamino)-phenyl]-isonicotinamide
2,6-Dichloro-N-[4-methyl-3-(4-pyridin-3-yl-thiazol-2-ylamino)-phenyl]-isonicotinamide
3-Phenyl-propynoic acid [4-methyl-3-(4-pyridin-3-yl-thiazol-2-ylamino)-phenyl]-amide
Cyclohexanecarboxylic acid [4-methyl-3-(4-pyridin-3-yl-thiazol-2-ylmethyl)-phenyl]-amide
5-[4-Methyl-3-(4-pyridin-3-yl-thiazol-2-ylamino)-phenylcarbamoyl]-pentanoic acid ethyl ester
1-Methyl-cyclohexanecarboxylic acid [4-methyl-3-(4-pyridin-3-yl-thiazol-2-ylmethyl)-phenyl]-amide
4-tert-Butyl-cyclohexanecarboxylic acid [4-methyl-3-(4-pyridin-3-yl-thiazol-2-ylamino)-phenyl]-amide
N-[4-Methyl-3-(4-pyridin-3-yl-thiazol-2-ylamino)-phenyl]-4-morpholin-4-yl-butyramide

Among the compounds of formula IV, the invention is particularly embodied by the compounds wherein X is a urea group, a —CO—NRR′ group, corresponding to the [3-(thiazol-2-ylamino)-phenyl]-urea family and the following formula: 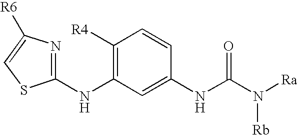
wherein Ra, Rb are independently chosen from Y-Z as defined above or
H or an organic group that can be selected for example from a linear or branched alkyl group containing from 1 to 10 carbon atoms optionally substituted with at least one heteroatom and/or bearing a pendant basic nitrogen functionality; a cycloalkyl, an aryl or heteroaryl group optionally substituted with a heteroatom, notably a halogen selected from I, Cl, Br and F or bearing a pendant basic nitrogen functionality; or a cycloalkyl, an aryl or heteroaryl group optionally substituted with a cycloalkyl, an aryl or heteroaryl group optionally substituted with a heteroatom, notably a halogen selected from I, Cl, Br and F or bearing a pendant basic nitrogen functionality;
a —SO2-R group wherein R is an alkyl, cycloalkyl, aryl or heteroaryl optionally substituted with an heteroatom, notably a halogen selected from I, Cl, Br and F or bearing a pendant basic nitrogen functionality; or a —CO—R or a —CO—NRR′ group, wherein R and R′ are independently chosen from H, an alkyl, a cycloalkyl, an aryl or heteroaryl group optionally substituted with at least one heteroatom, notably selected from I, Cl, Br and F, or bearing a pendant basic nitrogen functionality.

R⁴ is hydrogen, halogen or a linear or branched alkyl group containing from 1 to 10 carbon atoms, trifluoromethyl or alkoxy;

R⁶ is one of the following:

(i) an aryl group such as phenyl or a substituted variant thereof bearing any combination, at any one ring position, of one or more substituents such as halogen, alkyl groups containing from 1 to 10 carbon atoms, trifluoromethyl, and alkoxy;

(ii) a heteroaryl group such as a 2, 3, or 4-pyridyl group, which may additionally bear any combination of one or more substituents such as halogen, alkyl groups containing from 1 to 10 carbon atoms, trifluoromethyl and alkoxy;

(iii) a five-membered ring aromatic heterocyclic group such as for example 2-thienyl, 3-thienyl, 2-thiazolyl, 4-thiazolyl, 5-thiazolyl, which may additionally bear any combination of one or more substituents such as halogen, an alkyl group containing from 1 to 10 carbon atoms, trifluoromethyl, and alkoxy.

iv) H, a halogen selected from I, F, Cl or Br; NH2, NO2 or SO2-R, wherein R is a linear or branched alkyl group containing one or more group such as 1 to 10 carbon atoms, and optionally substituted with at least one heteroatom, notably a halogen selected from I, Cl, Br and F, and/or bearing a pendant basic nitrogen functionality.

### Example 1

1-(4-Methoxy-phenyl)-3-[4-methyl-3-(4-pyridin-3-yl-thiazol-2-ylamino)-phenyl]-urea
1-(4-Bromo-phenyl)-3-[4-methyl-3-(4-pyridin-3-yl-thiazol-2-ylamino)-phenyl]-urea
1-[4-Methyl-3-(4-pyridin-3-yl-thiazol-2-ylamino)-phenyl]-3-(4-trifluoromethyl-phenyl)-urea
1-(4-Fluoro-phenyl)-3-[4-methyl-3-(4-pyridin-3-yl-thiazol-2-ylamino)-phenyl]-urea
1-[4-Methyl-3-(4-pyridin-3-yl-thiazol-2-ylamino)-phenyl]-3-(3,4,5-trimethoxy-phenyl)-urea
4-{3-[4-Methyl-3-(4-pyridin-3-yl-thiazol-2-ylamino)-phenyl]-ureido}-benzoic acid ethyl ester
1-[4-Methyl-3-(4-pyridin-3-yl-thiazol-2-ylamino)-phenyl]-3-thiophen-2-yl-urea
1-Cyclohexyl-1-(N-Cyclohexyl-formamide)-3-[4-methyl-3-(4-pyridin-3-yl-thiazol-2-ylamino)-phenyl]-urea
1-(2,4-Dimethoxy-phenyl)-3-[4-methyl-3-(4-pyridin-3-yl-thiazol-2-ylamino)-phenyl]-urea
1-(2-Iodo-phenyl)-1-(N-(2-Iodo-phenyl)-formamide)-3-[4-methyl-3-(4-pyridin-3-yl-thiazol-2-ylamino)-phenyl]-urea
1-(3,5-Dimethyl-isoxazol-4-yl)-3-[4-methyl-3-(4-pyridin-3-yl-thiazol-2-ylamino)-phenyl]-urea
1-(2-Iodo-phenyl)-3-[4-methyl-3-(4-pyridin-3-yl-thiazol-2-yl amino)-phenyl]-urea
1-(4-Difluoromethoxy-phenyl)-3-[4-methyl-3-(4-pyridin-3-yl-thiazol-2-ylamino)-phenyl]-urea
1-(4-Dimethylamino-phenyl)-3-[4-methyl-3-(4-pyridin-3-yl-thiazol-2-ylamino)-phenyl]-urea
1-(2-Fluoro-phenyl)-3-[4-methyl-3-(4-pyridin-3-yl-thiazol-2-ylamino)-phenyl]-urea
1-(2-Chloro-phenyl)-3-[4-methyl-3-(4-pyridin-3-yl-thiazol-2-ylamino)-phenyl]-urea
1-(3-Fluoro-phenyl)-3-[4-methyl-3-(4-pyridin-3-yl-thiazol-2-ylamino)-phenyl]-urea
1-[4-Methyl-3-(4-pyridin-3-yl-thiazol-2-ylamino)-phenyl]-3-p-tolyl-urea
3-Bromo-N-[4-methyl-3-(4-pyridin-3-yl-thiazol-2-ylamino)-phenyl]-benzamide
3-Iodo-N-[4-methyl-3-(4-pyridin-3-yl-thiazol-2-ylamino)-phenyl]-benzamide
4-Hydroxymethyl-N-[4-methyl-3-(4-pyridin-3-yl-thiazol-2-ylamino)-phenyl]-benzamide
4-Amino-N-[4-methyl-3-(4-pyridin-3-yl-thiazol-2-ylamino)-phenyl]-benzamide
2-Iodo-N-[4-methyl-3-(4-pyridin-3-yl-thiazol-2-ylamino)-phenyl]-benzamide
4-Iodo-N-[4-methyl-3-(4-pyridin-3-yl-thiazol-2-ylamino)-phenyl]-benzamide
4-(3-{4-[4-Methyl-3-(4-pyridin-3-yl-thiazol-2-ylamino)-phenylcarbamoyl]-phenyl}-ureido)-benzoic acid ethyl ester
N-[4-Methyl-3-(4-pyridin-3-yl-thiazol-2-ylamino)-phenyl]-4-[3-(4-trifluoromethyl-phenyl)-ureido]-benzamide
4-[3-(4-Bromo-phenyl)-ureido]-N-[4-methyl-3-(4-pyridin-3-yl-thiazol-2-ylamino)-phenyl]-benzamide
4-Hydroxy-N-[4-methyl-3-(4-pyridin-3-yl-thiazol-2-ylamino)-phenyl]-benzamide
N-[4-Methyl-3-(4-pyridin-3-yl-thiazol-2-ylamino)-phenyl]-4-(3-thiophen-2-yl-ureido)-benzamide
4-[3-(3,5-Dimethyl-isoxazol-4-yl)-ureido]-N-[4-methyl-3-(4-pyridin-3-yl-thiazol-2-ylamino)-phenyl]-benzamide
4-[(4-Methoxy-phenyl)-ureido]-N-[4-methyl-3-(4-pyridin-3-yl-thiazol-2-ylamino)-phenyl]-benzamide
4-[3-(4-Difluoromethoxy-phenyl)-ureido]-N-[4-methyl-3-(4-pyridin-3-yl-thiazol-2-ylamino)-phenyl]-benzamide
Thiophene-2-sulfonic acid 4-[4-methyl-3-(4-pyridin-3-yl-thiazol-2-ylamino)-phenylcarbamoyl]-phenyl ester
4-Iodo-benzenesulfonic acid 4-[4-methyl-3-(4-pyridin-3-yl-thiazol-2-ylamino)-phenylcarbamoyl]-phenyl ester
N-[4-Methyl-3-(4-pyridin-3-yl-thiazol-2-ylamino)-phenyl]-4-(thiophene-2-sulfonylamino)-benzamide
3-Fluoro-N-[4-methyl-3-(4-pyridin-3-yl-thiazol-2-ylamino)-phenyl]-benzamide
N-[4-Methyl-3-(4-pyridin-3-yl-thiazol-2-ylamino)-phenyl]-4-pyridin-4-yl-benzamide
4-Dimethylamino-N-[4-methyl-3-(4-pyridin-3-yl-thiazol-2-ylamino)-phenyl]-benzamide
2-Fluoro-5-methyl-N-[4-methyl-3-(4-pyridin-3-yl-thiazol-2-ylamino)-phenyl]-benzamide
4-tert-Butyl-N-[4-methyl-3-(4-pyridin-3-yl-thiazol-2-ylamino)-phenyl]-benzamide
4-Isopropoxy-N-[4-methyl-3-(4-pyridin-3-yl-thiazol-2-ylmethyl)-phenyl]-benzamide
Benzo[1,3]dioxole-5-carboxylic acid [4-methyl-3-(4-pyridin-3-yl-thiazol-2-ylmethyl)-phenyl]-amide
N-[4-Methyl-3-(4-pyridin-3-yl-thiazol-2-yl amino)-phenyl]-3-(2-morpholin-4-yl-ethoxy)-benzamide
N-[4-Methyl-3-(4-pyridin-3-yl-thiazol-2-ylmethyl)-phenyl]-4-pyridin-4-yl-benzamide
3-Cyano-N-[4-methyl-3-(4-pyridin-3-yl-thiazol-2-ylamino)-phenyl]-benzamide
2-Fluoro-N-[4-methyl-3-(4-pyridin-3-yl-thiazol-2-ylamino)-phenyl]-3-trifluoromethyl-benzamide
3-Fluoro-benzenesulfonic acid 4-[4-methyl-3-(4-pyridin-3-yl-thiazol-2-ylamino)-phenylcarbamoyl]-phenyl ester
4-Aminomethyl-N-[4-methyl-3-(4-pyridin-3-yl-thiazol-2-ylamino)-phenyl]-benzamide
2-Fluoro-benzenesulfonic acid 4-[4-methyl-3-(4-pyridin-3-yl-thiazol-2-ylamino)-phenylcarbamoyl]-phenyl ester
3-Methoxy-N-[4-methyl-3-(4-pyridin-3-yl-thiazol-2-ylmethyl)-phenyl]-benzamide
4-(4-Methyl-piperazin-1-yl)-N-[4-methyl-3-(4-pyridin-3-yl-thiazol-2-ylmethyl)-phenyl]-benzamide
3-Methyl-N-[4-methyl-3-(4-pyridin-3-yl-thiazol-2-ylamino)-phenyl]-benzamide
Biphenyl-3-carboxylic acid [4-methyl-3-(4-pyridin-3-yl-thiazol-2-ylamino)-phenyl]-amide
N-[4-Methyl-3-(4-pyridin-3-yl-thiazol-2-ylamino)-phenyl]-3-trifluoromethyl-benzamide
N-[4-Methyl-3-(4-pyridin-3-yl-thiazol-2-ylamino)-phenyl]-4-pyrrolidin-1-ylmethyl-benzamide
4-[3-(2,4-Dimethoxy-phenyl)-ureido]-N-[4-methyl-3-(4-pyridin-3-yl-thiazol-2-ylamino)-phenyl]-benzamide
4-[3-(2-Iodo-phenyl)-ureido]-N-[4-methyl-3-(4-pyridin-3-yl-thiazol-2-ylamino)-phenyl]-benzamide
4-[3-(4-Fluoro-phenyl)-ureido]-N-[4-methyl-3-(4-pyridin-3-yl-thiazol-2-ylamino)-phenyl]-benzamide
3-Bromo-4-methyl-N-[4-methyl-3-(4-pyridin-3-yl-thiazol-2-ylamino)-phenyl]-benzamide
4-Fluoro-N-[4-methyl-3-(4-pyridin-3-yl-thiazol-2-ylamino)-phenyl]-benzamide
4-Cyano-N-[4-methyl-3-(4-pyridin-3-yl-thiazol-2-ylamino)-phenyl]-benzamide
4-Fluoro-N-[4-methyl-3-(4-pyridin-3-yl-thiazol-2-ylamino)-phenyl]-benzamide

### Example 2

4-(4-methyl-piperazin-1-ylmethyl)-N-[4-methyl-3-(4-pyridin-3-yl-thiazol-2-ylamino)-phenyl]-benzamide
3,5-Dibromo-4-(4-methyl-piperazin-1-ylmethyl)-N-[4-methyl-3-(4-pyridin-3-yl-thiazol-2-ylamino)-phenyl]-benzamide
4-Diethylaminomethyl-N-[4-methyl-3-(4-pyridin-3-yl-thiazol-2-ylamino)-phenyl]-benzamide
N-[4-Methyl-3-(4-pyridin-3-yl-thiazol-2-ylamino)-phenyl]-4-morpholin-4-ylmethyl-benzamide
4-Dipropylaminomethyl-N-[4-methyl-3-(4-pyridin-3-yl-thiazol-2-ylamino)-phenyl]-benzamide
N-[4-Methyl-3-(4-pyridin-3-yl-thiazol-2-ylamino)-phenyl]-4-piperidin-1-ylmethyl-benzamide
4-[(Diisopropylamino)-methyl]-N-[4-methyl-3-(4-pyridin-3-yl-thiazol-2-ylamino)-phenyl]-benzamide
{4-[4-Methyl-3-(4-pyridin-3-yl-thiazol-2-ylamino)-phenylcarbamoyl]-benzyl}-carbamic acid tert-butyl ester
3-Fluoro-4-(4-methyl-piperazin-1-ylmethyl)-N-[4-methyl-3-(4-pyridin-3-yl-thiazol-2-ylamino)-phenyl]-benzamide
4-(4-Methyl-piperazin-1-ylmethyl)-N-[4-methyl-3-(4-pyridin-3-yl-thiazol-2-ylmethyl)-phenyl]-3-trifluoromethyl-benzamide
2,3,5,6-Tetrafluoro-4-(4-methyl-piperazin-1-ylmethyl)-N-[4-methyl-3-(4-pyridin-3-yl-thiazol-2-ylamino)-phenyl]-benzamide
N-{3-[4-(4-Fluoro-phenyl)-thiazol-2-ylamino]-4-methyl-phenyl}-4-(4-methyl-piperazin-1-ylmethyl)-benzamide
3-Bromo-4-(4-methyl-piperazin-1-ylmethyl)-N-[4-methyl-3-(4-pyridin-3-yl-thiazol-2-ylamino)-phenyl]-benzamide
3-Chloro-4-(4-methyl-piperazin-1-ylmethyl)-N-[4-methyl-3-(4-pyridin-3-yl-thiazol-2-ylamino)-phenyl]-benzamide
4-(4-Methyl-piperazin-1-ylmethyl)-N-[4-methyl-3-(4-pyridin-4-yl-thiazol-2-ylamino)-phenyl]-benzamide
N-{3-[4-(4-Cyano-phenyl)-thiazol-2-ylamino]-4-ethyl-phenyl}-4-(4-methyl-piperazin-1-ylmethyl)-benzamide
4-[1-(4-Methyl-piperazin-1-yl)-ethyl]-N-[4-methyl-3-(4-pyridin-3-yl-thiazol-2-ylmethyl)-phenyl]-benzamide
4-(1-Methoxy-ethyl)-N-[4-methyl-3-(4-pyridin-3-yl-thiazol-2-ylmethyl)-phenyl]-benzamide
N-4-Methyl-3-[4-(4-methyl-pyridin-3-yl)-thiazol-2-ylamino]-phenyl-4-(4-methyl-piperazin-1-ylmethyl)-benzamide
3-Iodo-4-(4-methyl-piperazin-1-ylmethyl)-N-[4-methyl-3-(4-pyridin-3-yl-thiazol-2-ylmethyl)-phenyl]-benzamide
N-[4-Methyl-3-(4-pyridin-3-yl-thiazol-2-ylamino)-phenyl]-4-[3-(4-trifluoromethyl-phenyl)-ureidomethyl]-benzamide
3,5-Dibromo-N-[4-methyl-3-(4-pyridin-3-yl-thiazol-2-ylamino)-phenyl]-4-[(3-morpholin-4-yl-propylamino)-methyl]-benzamide
3,5-Dibromo-N-[4-methyl-3-(4-pyridin-3-yl-thiazol-2-ylamino)-phenyl]-4-piperidin-1-ylmethyl-benzamide
4-(4-Methyl-piperazin-1-ylmethyl)-N-[4-methyl-3-(4-pyridin-2-yl-thiazol-2-ylamino)-phenyl]-benzamide
N-{3-[4-(3-Fluoro-phenyl)-thiazol-2-ylamino]-4-methyl-phenyl}-4-(4-methyl-piperazin-1-ylmethyl)-benzamide
N-{3-[4-(2-Fluoro-phenyl)-thiazol-2-ylamino]-4-methyl-phenyl}-4-(4-methyl-piperazin-1-ylmethyl)-benzamides

### Example 3

3-Dimethylamino-N-[4-methyl-3-(4-pyridin-3-yl-thiazol-2-ylamino)-phenyl]-benzamide
3-(4-Methyl-piperazin-1′-yl)-N-[4-methyl-3-(4-pyridin-3-yl-thiazol-2-ylamino)-phenyl]-benzamide
N-[4-Methyl-3-(4-pyridin-3-yl-thiazol-2-ylamino)-phenyl]-3-morpholin-4-yl-benzamide

Among the compounds of formula IV, the invention is particularly embodied by the compounds wherein X is a —OR group, corresponding to the family [3-(Thiazol-2-ylamino)-phenyl]-carbamate and the following formula IV-6 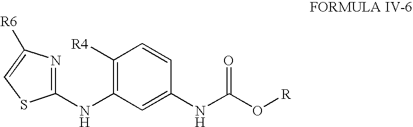
wherein R is independently chosen from an organic group that can be selected for example from a linear or branched alkyl group containing from 1 to 10 carbon atoms optionally substituted with at least one heteroatom and/or bearing a pendant basic nitrogen functionality; a cycloalkyl, an aryl or heteroaryl group optionally substituted with an heteroatom, notably a halogen selected from I, Cl, Br and F and/or bearing a pendant basic nitrogen functionality; or a cycloalkyl, an aryl or heteroaryl group optionally substituted with a cycloalkyl, an aryl or heteroaryl group optionally substituted with a heteroatom, notably a halogen selected from I, Cl, Br and F and/or bearing a pendant basic nitrogen functionality;
R4 and R6 are as defined above.

In still another preferred embodiment, the invention contemplated the method mentioned above, wherein said c-kit inhibitor is selected from 2-aminoaryloxazoles of formula X: 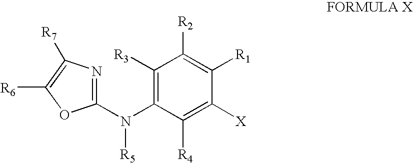
wherein substituents R1-R7 and X are defined as follows:

R1, R2, R3 and R4 each independently are selected from hydrogen, halogen (selected from F, Cl, Br or I), a linear or branched alkyl group containing from 1 to 10 carbon atoms and optionally substituted with one or more hetereoatoms such as halogen (selected from F, Cl, Br or I), oxygen, and nitrogen, the latter optionally in the form of a pendant basic nitrogen functionality; as well as trifluoromethyl, C₁₋₆alkyloxy, amino, C₁₋₆alkylamino, di(C₁₋₆alkyl)amino, carboxyl, cyano, nitro, formyl, hydroxy, and CO—R, COO—R, CONH—R, SO2-R, and SO2NH—R wherein R is a linear or branched alkyl group containing from 1 to 10 carbon atoms and optionally substituted with at least one heteroatom, notably a halogen (selected from F, Cl, Br or I), oxygen, and nitrogen, the latter optionally in the form of a pendant basic nitrogen functionality.

R5 is one of the following:

(i) hydrogen, or

(ii) a linear or branched alkyl group containing from 1 to 10 carbon atoms and optionally substituted with one or more hetereoatoms such as halogen (selected from F, Cl, Br or I), oxygen, and nitrogen, the latter optionally in the form of a pendant basic nitrogen functionality, or

(iii) CO—R8 or COOR8 or CONHR8 or SO2R8 wherein R8 may be

a linear or branched alkyl group containing from 1 to 10 carbon atoms and optionally substituted with one or more hetereoatoms such as halogen (selected from F, Cl, Br or I), oxygen, and nitrogen, the latter optionally in the form of a pendant basic nitrogen functionality, or
an aryl group such as phenyl or a substituted variant thereof bearing any combination, at any one ring position, of one or more substituents such as halogen (selected from F, Cl, Br or I), alkyl groups containing from 1 to 10 carbon atoms and optionally substituted with one or more hetereoatoms such as halogen (selected from F, Cl, Br or I), oxygen, and nitrogen, the latter optionally in the form of a pendant basic nitrogen functionality; as well as trifluoromethyl, C₁₋₆alkyloxy, carboxyl, cyano, nitro, formyl, hydroxy, C₁₋₆alkylamino, di(C₁₋₆alkyl)amino, and amino, the latter nitrogen substituents optionally in the form of a pendant basic nitrogen functionality; as well as CO—R, COO—R, CONH—R, SO2-R, and SO2NH—R wherein R is a linear or branched alkyl group containing from 1 to 10 carbon atoms and optionally substituted with at least one heteroatom, notably a halogen (selected from F, Cl, Br or I), oxygen, and nitrogen, the latter optionally in the form of a pendant basic nitrogen functionality, or
a heteroaryl group such as a pyridyl, pyrimidinyl, pyrazinyl, pyridazinyl, thienyl, thiazolyl, imidazolyl, pyrazolyl, pyrrolyl, furanyl, oxazolyl, isoxazolyl, triazolyl, tetrazolyl, indolyl, benzimidazole, quinolinyl group, which may additionally bear any combination, at any one ring position, of one or more substituents such as halogen (selected from F, Cl, Br or I), alkyl groups containing from 1 to 10 carbon atoms and optionally substituted with one or more hetereoatoms such as halogen (selected from F, Cl, Br or I), oxygen, and nitrogen, the latter optionally in the form of a pendant basic nitrogen functionality; as well as trifluoromethyl, C₁₋₆alkyloxy, carboxyl, cyano, nitro, formyl, hydroxy, C₁₋₆alkylamino, di(C₁₋₆alkyl)amino, and amino, the latter nitrogen substituents optionally in the form of a basic nitrogen functionality; as well as CO—R, COO—R, CONH—R, SO2-R, and SO2NH—R wherein R is a linear or branched alkyl group containing from 1 to 10 carbon atoms and optionally substituted with at least one heteroatom, notably a halogen (selected from F, Cl, Br or I), oxygen, and nitrogen, the latter optionally in the form of a pendant basic nitrogen functionality.

R6 and R7 each independently are selected from:

i) hydrogen, a halogen (selected from F, Cl, Br or I), or

ii) an alkyl¹ group defined as a linear, branched or cycloalkyl group containing from 1 to 10 carbon atoms and optionally substituted with one or more hetereoatoms such as halogen (selected from F, Cl, Br or I), oxygen, and nitrogen (the latter optionally in the form of a pendant basic nitrogen functionality); as well as trifluoromethyl, carboxyl, cyano, nitro, formyl; as well as CO—R, COO—R, CONH—R, SO2-R, and SO2NH—R wherein R is a linear or branched alkyl group containing 1 to 10 carbon atoms and optionally substituted with at least one heteroatom, notably a halogen (selected from F, Cl, Br or I), oxygen, and nitrogen, the latter optionally in the form of a pendant basic nitrogen functionality; as well as a cycloalkyl or aryl or heteroaryl group optionally substituted by a pendant basic nitrogen functionality, or

(iii) an aryl¹ group defined as phenyl or a substituted variant thereof bearing any combination, at any one ring position, of one or more substituents such as

halogen (selected from I, F, Cl or Br);
an alkyl¹ group;
a cycloalkyl, aryl or heteroaryl group optionally substituted by a pendant basic nitrogen functionality;
trifluoromethyl, O-alkyl¹, carboxyl, cyano, nitro, formyl, hydroxy, NH-alkyl¹, N(alkyl¹)(alkyl¹), and amino, the latter nitrogen substituents optionally in the form of a basic nitrogen functionality;
NHCO—R or NHCOO—R or NHCONH—R or NHSO2-R or NHSO2NH—R or CO—R or COO—R or CONH—R or SO2-R or SO2NH—R wherein R corresponds to hydrogen, alkyl¹, aryl or heteroaryl, or
   (iv) a heteroaryl¹ group defined as a pyridyl, pyrimidinyl, pyrazinyl, pyridazinyl, thienyl, thiazolyl, imidazolyl, pyrazolyl, pyrrolyl, furanyl, oxazolyl, isoxazolyl, triazolyl, tetrazolyl, indolyl, benzimidazole, quinolinyl group, which may additionally bear any combination, at any one ring position, of one or more substituents such as
halogen (selected from F, Cl, Br or I);
an alkyl¹ group;
a cycloalkyl, aryl or heteroaryl group optionally substituted by a pendant basic nitrogen functionality,
trifluoromethyl, O-alkyl¹, carboxyl, cyano, nitro, formyl, hydroxy, NH-alkyl¹, N(alkyl¹)(alkyl¹), and amino, the latter nitrogen substituents optionally in the form of a basic nitrogen functionality;
NHCO—R or NHCOO—R or NHCONH—R or NHSO2-R or NHSO2NH—R or CO—R or COO—R or CONH—R or SO2-R or SO2NH—R wherein R corresponds to hydrogen, alkyl¹, or
   (v) an O-aryl¹, or NH-aryl¹, or O-heteroaryl¹ or NH-heteroaryl¹ group
   (vi) trifluoromethyl, O-alkyl¹, carboxyl, cyano, nitro, formyl, hydroxy, NH-alkyl¹, N(alkyl¹)(alkyl¹), and amino, the latter nitrogen substituents optionally in the form of a basic nitrogen functionality, or
   (vi) NHCO—R or NHCOO—R or NHCONH—R or NHSO2-R or NHSO2NH—R or CO—R or COO—R or CONH—R or SO2-R or SO2NH—R wherein R corresponds to hydrogen, alkyl¹, aryl or heteroaryl.

X is:

—NR9R10, wherein R9 and/or R10 are hydrogen or:

i) an alkyl¹ group, CF3 or ii) an aryl¹, heteroaryl¹ or cycloalkyl group optionally substituted by a pendant basic nitrogen functionality, or

iii) a CO—R, COO—R, CON—RR′ or SO2-R, where R and R′ are a hydrogen, alkyl¹, aryl¹ or heteroaryl¹, optionally substituted by a pendant basic nitrogen functionality;

or:

—CO—NR9R10, wherein R9 and/or R10 are hydrogen or:

i) an alkyl¹ group, CF3 or

ii) an aryl¹, heteroaryl¹ or cycloalkyl group optionally substituted by a pendant basic nitrogen functionality.

Such compound may be selected from N-Aminoalkyl-N′-oxazol-2-yl-benzene-1,3-diamines of the following formula: 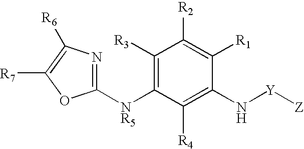
wherein R5=H, Y is a linear or branched alkyl group containing from 1 to 10 carbon atoms and Z represents an aryl or a heteroaryl group, optionally substituted by a pendant basic nitrogen functionality.

For example, it is the 4-{[4-Methyl-3-(4-pyridin-3-yl-oxazol-2-ylamino)-phenylamino]-methyl}-benzoic acid methyl ester.

The above 2-aminoaryloxazoles compounds may have the formula XI:

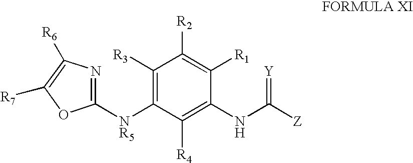

Wherein R5 is H, Y is selected from O, S and Z corresponds to H, alkyl, or NRR′, wherein R and R′ are independently chosen from H or alkyl¹ or aryl¹ or heteroaryl¹, optionally substituted by a pendant basic nitrogen functionality, for example: 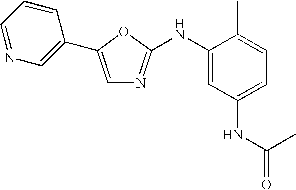
or a compound of formula XI-1: 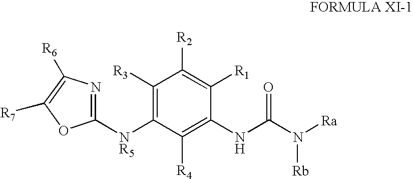
wherein Ra, Rb are independently chosen from H or alkyl¹ or aryl¹ or heteroaryl¹, optionally substituted by a pendant basic nitrogen functionality, for example: 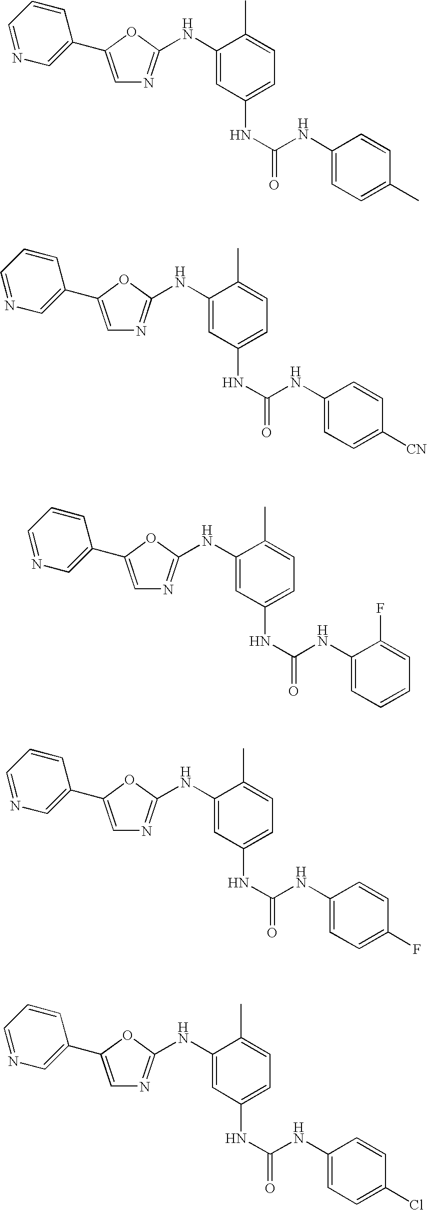
or a compound of formula XI-2: 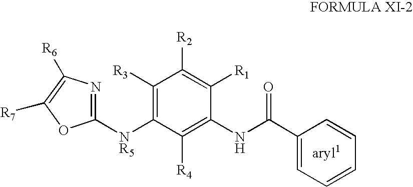
wherein R5=H, Z is an aryl¹ group, aryl¹ being selected from:
a phenyl or a substituted variant thereof bearing any combination, at any one ring position, of one or more substituents such as
halogen (selected from I, F, Cl or Br);
an alkyl¹ group;
a cycloalkyl, aryl or heteroaryl group optionally substituted by a pendant basic nitrogen functionality;
trifluoromethyl, O-alkyl¹, carboxyl, cyano, nitro, formyl, hydroxy, NH-alkyl¹, N(alkyl¹)(alkyl¹), and amino, the latter nitrogen substituents optionally in the form of a basic nitrogen functionality;
   NHCO—R or NHCOO—R or NHCONH—R or NHSO2-R or NHSO2NH—R or CO—R or COO—R or CONH—R or SO2-R or SO2NH—R wherein R corresponds to hydrogen, alkyl¹, aryl or heteroaryl, for example 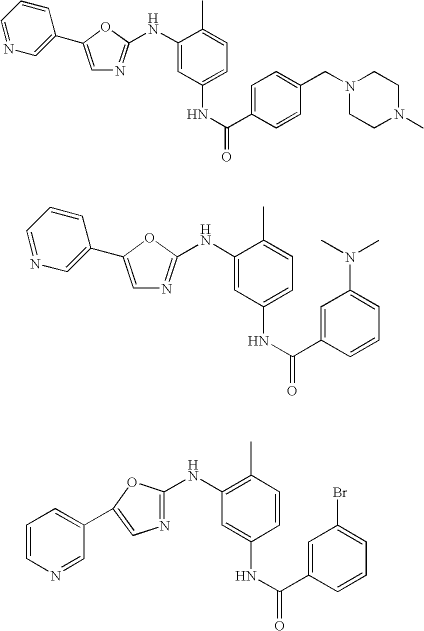
   or a compound of formula XI-3: 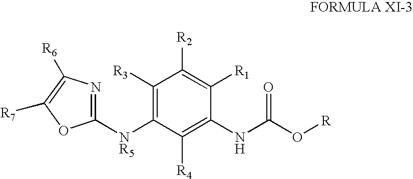
   wherein R5=H and R is independently alkyl¹, aryl¹ or heteroaryl¹ as defined above.
   Examples of Compounds of Formula X:
4-{[4-Methyl-3-(4-pyridin-3-yl-oxazol-2-ylamino)-phenylamino]-methyl}-benzoic acid methyl ester
4-Methyl-N1-(5-pyridin-3-yl-oxazol-2-yl)-N3-(5-pyridin-4-yl-oxazol-2-yl)-benzene-1,3-diamine m.p.
4-Methyl-N1-(5-phenyl-oxazol-2-yl)-N3-(5-pyridin-4-yl-oxazol-2-yl)-benzene-1,3-diamine
4-Methyl-N1-(5-phenyl-[1,3,4]oxadiazol-2-yl)-N3-(5-pyridin-4-yl-oxazol-2-yl)-benzene-1,3-diamine
N1-Benzooxazol-2-yl-4-methyl-N3-(5-pyridin-4-yl-oxazol-2-yl)-benzene-1,3-diamine
N-[4-Methyl-3-(5-pyridin-4-yl-oxazol-2-ylamino)-phenyl]-C-phenyl-methanesulfon-amide
N-[4-Methyl-3-(5-pyridin-3-yl-oxazol-2-ylamino)-phenyl]-acetamide
2-Cyano-N-[4-methyl-3-(5-pyridin-4-yl-oxazol-2-ylamino)-phenyl]-acetamide
2-Ethoxy-N-[4-methyl-3-(5-pyridin-3-yl-oxazol-2-ylamino)-phenyl]-acetamide
3-Methoxy-N-[4-methyl-3-(5-pyridin-3-yl-oxazol-2-ylamino)-phenyl]-propionamide
1-(4-Cyano-phenyl)-3-[4-methyl-3-(5-pyridin-3-yl-oxazol-2-ylamino)-phenyl]-urea
1-(4-Fluoro-phenyl)-3-[4-methyl-3-(5-pyridin-3-yl-oxazol-2-ylamino)-phenyl]-urea
1-(2-Fluoro-phenyl)-3-[4-methyl-3-(5-pyridin-3-yl-oxazol-2-ylamino)-phenyl]-urea
1-[4-Methyl-3-(5-pyridin-3-yl-oxazol-2-ylamino)-phenyl]-3-(4-trifluoromethyl-phenyl)-urea
1-(4-Chloro-phenyl)-3-[4-methyl-3-(5-pyridin-3-yl-oxazol-2-ylamino)-phenyl]-urea
1-[4-Methyl-3-(5-phenyl-oxazol-2-ylamino)-phenyl]-3-(3-trifluoromethyl-phenyl)-urea
1-(4-Cyano-phenyl)-3-[4-methyl-3-(5-pyridin-3-yl-oxazol-2-ylamino)-phenyl]-thiourea
1-(4-Cyano-phenyl)-3-[4-methyl-3-(5-pyridin-4-yl-oxazol-2-ylamino)-phenyl]-thiourea
(2-{2-Methyl-5-[3-(4-trifluoromethyl-phenyl)-ureido]-phenylamino}-oxazol-5-yl)-acetic acid ethyl ester
1-Benzyl-3-[4-methyl-3-(5-pyridin-4-yl-oxazol-2-ylamino)-phenyl]-thiourea
4-(4-Methyl-piperazin-1-ylmethyl)-N-[4-methyl-3-(5-pyridin-3-yl-oxazol-2-ylamino)-phenyl]-benzamide
3-Dimethylamino-N-[4-methyl-3-(5-pyridin-3-yl-oxazol-2-ylamino)-phenyl]-benzamide
3-Bromo-N-[4-methyl-3-(5-pyridin-3-yl-oxazol-2-ylamino)-phenyl]-benzamide
N-[4-Methoxy-3-(5-pyridin-3-yl-oxazol-2-ylamino)-phenyl]-3-trifluoromethyl-benzamide
4-(3-Dimethylamino-propylamino)-N-[4-methyl-3-(5-pyridin-3-yl-oxazol-2-ylamino)-phenyl]-3-trifluoromethyl-benzamide
N-[4-Fluoro-3-(5-pyridin-3-yl-oxazol-2-ylamino)-phenyl]-3-trifluoromethyl-benzamide
1H-Indole-6-carboxylic acid [4-methyl-3-(5-pyridin-4-yl-oxazol-2-ylamino)-phenyl]-amide
3-Isopropoxy-N-[4-methyl-3-(5-pyridin-4-yl-oxazol-2-ylamino)-phenyl]-benzamide
N-[4-Methyl-3-(5-pyridin-2-yl-oxazol-2-ylamino)-phenyl]-3-trifluoromethyl-benzamide
3,5-Dimethoxy-N-[4-methyl-3-(5-pyridin-4-yl-oxazol-2-ylamino)-phenyl]-benzamide
N-[3-(5-Pyridin-3-yl-oxazol-2-ylamino)-phenyl]-3-trifluoromethyl-benzamide
N-[4-Methyl-3-(5-phenyl-oxazol-2-ylamino)-phenyl]-3-trifluoromethyl-benzamide
3-Fluoro-4-(4-methyl-piperazin-1-ylmethyl)-N-[4-methyl-3-(5-pyridin-3-yl-oxazol-2-ylamino)-phenyl]-benzamide
N-[4-Chloro-3-(5-pyridin-3-yl-oxazol-2-ylamino)-phenyl]-3-trifluoromethyl-benzamide
N-[4-Methyl-3-(5-pyridin-3-yl-oxazol-2-ylamino)-phenyl]-terephthalamide
5-Methyl-isoxazole-4-carboxylic acid [4-methyl-3-(5-pyridin-4-yl-oxazol-2-ylamino)-phenyl]-amide
4-Cyano-N-[4-methyl-3-(5-pyridin-4-yl-oxazol-2-ylamino)-phenyl]-benzamide
N-[4-Methyl-3-(5-pyridin-3-yl-oxazol-2-ylamino)-phenyl]-isonicotinamide
N-[4-Methyl-3-(4-pyridin-3-yl-oxazol-2-ylamino)-phenyl]-3-trifluoromethyl-benzamide
[4-Methyl-3-(5-pyridin-3-yl-oxazol-2-ylamino)-phenyl]-carbamic acid isobutyl ester
(5-Isobutoxycarbonylamino-2-methyl-phenyl)-(5-pyridin-3-yl-oxazol-2-yl)-carbamic acid isobutyl ester
[4-Methyl-3-(5-pyridin-4-yl-oxazol-2-ylamino)-phenyl]-carbamic acid isobutyl ester
N-[4-Methyl-3-(5-pyridin-4-yl-oxazol-2-ylamino)-phenyl]-2-m-tolyl-acetamide
2-(4-Fluoro-phenyl)-N-[4-methoxy-3-(5-pyridin-4-yl-oxazol-2-ylamino)-phenyl]-acetamide
2-(2,4-Difluoro-phenyl)-N-[4-methyl-3-(5-phenyl-oxazol-2-ylamino)-phenyl]-acetamide
2-(3-Bromo-phenyl)-N-[4-methyl-3-(5-pyridin-2-yl-oxazol-2-ylamino)-phenyl]-acetamide
3-(4-Fluoro-phenyl)-N-[4-methyl-3-(5-pyridin-4-yl-oxazol-2-ylamino)-phenyl]-propionamide
N-{3-[5-(4-Cyano-phenyl)-oxazol-2-ylamino]-4-methyl-phenyl}-2-(2,4-difluoro-phenyl)-acetamide
4-Methyl-pentanoic acid [4-methyl-3-(5-pyridin-3-yl-oxazol-2-ylamino)-phenyl]-amide
N-[4-Methyl-3-(5-pyridin-3-yl-oxazol-2-ylamino)-phenyl]-2-piperazin-1-yl-acetamide
N-[4-Methyl-3-(5-pyridin-3-yl-oxazol-2-ylamino)-phenyl]-3-piperazin-1-yl-propionamide
2-(2,6-Dichloro-phenyl)-N-[4-methyl-3-(5-pyridin-4-yl-oxazol-2-ylamino)-phenyl]-acetamide
N-[4-Methyl-3-(5-pyridin-3-yl-oxazol-2-ylamino)-phenyl]-3-pyrrolidin-1-yl-propionamide
N-[4-Methoxy-3-(5-pyridin-4-yl-oxazol-2-ylamino)-phenyl]-2-(4-trifluoromethyl-phenyl)-acetamide
2-(4-Methoxy-phenyl)-N-[4-methyl-3-(5-pyridin-4-yl-oxazol-2-ylamino)-phenyl]-acetamide
N-(4-Cyano-phenyl)-4-methyl-3-(5-pyridin-3-yl-oxazol-2-ylamino)-benzamide
N-(3-Dimethylamino-phenyl)-4-methyl-3-(5-pyridin-4-yl-oxazol-2-ylamino)-benzamide
N-(2-Dimethylamino-ethyl)-4-methyl-3-(5-pyridin-3-yl-oxazol-2-ylamino)-benzamide
N-(3-Fluoro-4-methyl-phenyl)-4-methyl-3-(5-pyridin-4-yl-oxazol-2-ylamino)-benzamide
N-(3-Chloro-phenyl)-4-methyl-3-(5-pyridin-3-yl-oxazol-2-ylamino)-benzamide
N-Benzyl-4-methyl-3-(5-pyridin-4-yl-oxazol-2-ylamino)-benzamide
N-(4-Methoxy-benzyl)-4-methyl-3-(5-pyridin-4-yl-oxazol-2-ylamino)-benzamide
[4-Methyl-3-(5-pyridin-4-yl-oxazol-2-ylamino)-phenyl]-morpholin-4-yl-methanone
[4-Methyl-3-(5-pyridin-4-yl-oxazol-2-ylamino)-phenyl]-piperazin-1-yl-methanone
N-(4-Fluoro-phenyl)-2-[4-methyl-3-(5-pyridin-4-yl-oxazol-2-ylamino)-phenyl]-acetamide
   Process for Manufacturing a Compound of Formula III Depicted Above.

This entails the condensation of a substrate of general formula 10 with a thiourea of the type 11.

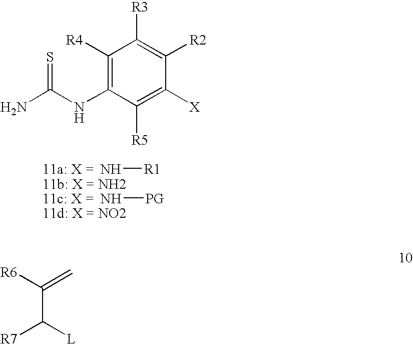

Substituent “L” in formula 10 is a nucleofugal leaving group in nucleophilic substitution reactions (for example, L can be selected from chloro, bromo, iodo, toluenesulfonyloxy, methanesulfonyloxy, trifluoromethanesulfonyloxy, etc., with L being preferentially a bromo group).

Group R1 in formula 11a corresponds to group R1 as described in formula III.

Group “PG” in formula 11c is a suitable protecting group of a type commonly utilized by the person skilled in the art.

The reaction of 10 with 1a-d leads to a thiozole-type product of formula 12a-d.

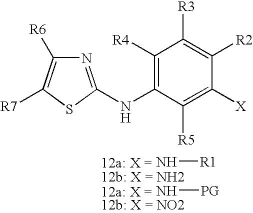

Formula 12a is the same as formula I. Therefore, R1 in 12a corresponds to R1 in formula III.

Formula 12b describes a precursor to compounds of formula III which lack substituent R1. Therefore, in a second phase of the synthesis, substituent R1 is connected to the free amine group in 12b, leading to the complete structure embodied by formula III:
12b+“R1”→III

The introduction of R1, the nature of which is as described on page 3 for the general formula III, is achieved by the use of standard reactions that are well known to the person skilled in the art, such as alkylation, acylation, sulfonylation, formation of ureas, etc.

Formula 12c describes an N-protected variant of compound 12b. Group “PG” in formula 12c represents a protecting group of the type commonly utilized by the person skilled in the art. Therefore, in a second phase of the synthesis, group PG is cleaved to transform compound 12c into compound 12b. Compound 12b is subsequently advanced to structures of formula I as detailed above.

Formula 12d describes a nitro analogue of compound 12b. In a second phase of the synthesis, the nitro group of compound 12d is reduced by any of the several methods utilized by the person skilled in the art to produce the corresponding amino group, namely compound 12b. Compound 12b thus obtained is subsequently advanced to structures of formula III as detailed above.

Examples of compound synthesis is found in our previous applications WO 2004/014903 and U.S. 60/513,214, incorporated herein by reference.

In a further embodiment, c-kit inhibitors as mentioned above are inhibitors of wild type or mutant activated c-kit. In this regard, the invention contemplates a method for treating patients exposed to chemical or biological weapons as defined above comprising administering to a human in need of such treatment a compound that is a selective, potent and non toxic inhibitor of c-kit obtainable by a screening method which comprises:

a) bringing into contact (i) activated c-kit and (ii) at least one compound to be tested; under conditions allowing the components (i) and (ii) to form a complex,

b) selecting compounds that inhibit activated c-kit,

c) testing and selecting a subset of compounds identified in step b), which are unable to promote death of IL-3 dependent cells cultured in presence of IL-3.

This screening method can further comprise the step consisting of testing and selecting a subset of compounds identified in step b) that are inhibitors of mutant activated c-kit (for example in the transphosphorylase domain), which are also capable of inhibiting SCF-activated c-kit wild. Alternatively, in step a) activated c-kit is SCF-activated c-kit wild.

A best mode for practicing this method consists of testing putative inhibitors at a concentration above 10 μM in step a). In step c), IL-3 is preferably present in the culture media of IL-3 dependent cells at a concentration comprised between 0.5 and 10 ng/ml, preferably between 1 to 5 ng/ml. These screening may be performed following our previous application WO 03/003006, which is incorporated herein by reference.

More particularly, the above compounds are useful for preventing or postponing the onset or development of inflammation and tissue damages of patients exposed to chemical or biological weapons.

The pharmaceutical compositions utilized in this invention may be administered by any number of routes including, but not limited to, oral, intravenous, intramuscular, intra-arterial, intramedullary, intrathecal, intraventricular, transdermal, subcutaneous, intraperitoneal, intranasal, enteral, sublingual, or rectal means.

In addition to the active ingredients, these pharmaceutical compositions may contain suitable pharmaceutically-acceptable carriers comprising excipients and auxiliaries which facilitate processing of the active compounds into preparations which can be used pharmaceutically. Further details on techniques for formulation and administration may be found in the latest edition of Remington's Pharmaceutical Sciences (Maack Publishing Co., Easton, Pa.).

Pharmaceutical compositions for oral administration can be formulated using pharmaceutically acceptable carriers well known in the art in dosages suitable for oral administration. Such carriers enable the pharmaceutical compositions to be formulated as tablets, pills, dragees, capsules, liquids, gels, syrups, slurries, suspensions, and the like, for ingestion by the patient.

More particularly, the invention relates to a pharmaceutical composition intended for oral administration.

However, it will be understood that in case of skin contact resulting in skin burns or inflammation as well as irritation, a topical composition may also be administered.

In this regard, the composition according to the invention comprises any ingredient commonly used in dermatology and cosmetic. It may comprise at least one ingredient selected from hydrophilic or lipophilic gelling agents, hydrophilic or lipophilic active agents, preservatives, emollients, viscosity enhancing polymers, humectants, surfactants, preservatives, antioxidants, solvents, and fillers, antioxidants, solvents, perfumes, fillers, screening agents, bactericides, odor absorbers and coloring matter.

As oils which can be used in the invention, mineral oils (liquid paraffin), vegetable oils (liquid fraction of shea butter, sunflower oil), animal oils, synthetic oils, silicone oils (cyclomethicone) and fluorinated oils may be mentioned. Fatty alcohols, fatty acids (stearic acid) and waxes (paraffin, carnauba, beeswax) may also be used as fatty substances.

As emulsifiers which can be used in the invention, glycerol stearate, polysorbate 60 and the PEG-6/PEG-32/glycol stearate mixture are contemplated.

As hydrophilic gelling agents, carboxyvinyl polymers (carbomer), acrylic copolymers such as acrylate/alkylacrylate copolymers, polyacrylamides, polysaccharides such as hydroxypropylcellulose, clays and natural gums may be mentioned, and as lipophilic gelling agents, modified clays such as bentones, metal salts of fatty acids such as aluminum stearates and hydrophobic silica, or alternatively ethylcellulose and polyethylene may be mentioned.

As hydrophilic active agents, proteins or protein hydrolysates, amino acids, polyols, urea, allantoin, sugars and sugar derivatives, vitamins, starch and plant extracts, in particular those of Aloe vera may be used.

As lipophilic active, agents, retinol (vitamin A) and its derivatives, tocopherol (vitamin E) and its derivatives, essential fatty acids, ceramides and essential oils may be used. These agents add extra moisturizing or skin softening features when utilized.

In addition, a surfactant can be included in the composition so as to provide deeper penetration of the ingredients and of the tyrosine kinase inhibitor.

Among the contemplated ingredients, the invention embraces penetration enhancing agents selected for example from the group consisting of mineral oil, water, ethanol, triacetin, glycerin and propylene glycol; cohesion agents selected for example from the group consisting of polyisobutylene, polyvinyl acetate and polyvinyl alcohol, and thickening agents.

Chemical methods of enhancing topical absorption of drugs are well known in the art. For example, compounds with penetration enhancing properties include sodium lauryl sulfate (Dugard, P. H. and Sheuplein, R. J., “Effects of Ionic Surfactants on the Permeability of Human Epidermis: An Electrometric Study,” J. Ivest. Dermatol., V. 60, pp. 263-69, 1973), lauryl amine oxide (Johnson et. al., U.S. Pat. No. 4,411,893), azone (Rajadhyaksha, U.S. Pat. Nos. 4,405,616 and 3,989,816) and decylmethyl sulfoxide (Sekura, D. L. and Scala, J., “The Percutaneous Absorption of Alkylmethyl Sulfides,” Pharmacology of the Skin, Advances In Biology of Skin, (Appleton-Century Craft) V. 12, pp. 257-69, 1972). It has been observed that increasing the polarity of the head group in amphoteric molecules increases their penetration-enhancing properties but at the expense of increasing their skin irritating properties (Cooper, E. R. and Berner, B., “Interaction of Surfactants with Epidermal Tissues: Physiochemical Aspects,” Surfactant Science Series, V. 16, Reiger, M. M. ed. (Marcel Dekker, Inc.) pp. 195-210, 1987).

Regarding Anthrax, it will be understood that the pharmaceutical composition may be intended for administration with aerosolized or intranasal formulation to target areas of a patient's respiratory tract.

Devices and methodologies for delivering aerosolized bursts of a formulation of a drug is disclosed in U.S. Pat. No. 5,906,202. Formulations are preferably solutions, e.g. aqueous solutions, ethanoic solutions, aqueous/ethanoic solutions, saline solutions, colloidal suspensions and microcrystalline suspensions. For example aerosolized particles comprise the active ingredient mentioned above and a carrier, (e.g., a pharmaceutically active respiratory drug and carrier) which are formed upon forcing the formulation through a nozzle which nozzle is preferably in the form of a flexible porous membrane. The particles have a size which is sufficiently small such that when the particles are formed they remain suspended in the air for a sufficient amount of time such that the patient can inhale the particles into the patient's lungs.

The invention encompasses systems described in U.S. Pat. No. 5,556,611:
liquid gas systems (a liquefied gas is used as propellent gas (e.g. low-boiling FCHC or propane, butane) in a pressure container,
suspension aerosol (the active substance particles are suspended in solid form in the liquid propellent phase),
pressurized gas system (a compressed gas such as nitrogen, carbon dioxide, dinitrogen monoxide, air is used.

Thus, according to the invention the pharmaceutical preparation is made in that the active substance is dissolved or dispersed in a suitable nontoxic medium and said solution or dispersion atomized to an aerosol, i.e. distributed extremely finely in a carrier gas. This is technically possible for example in the form of aerosol propellent gas packs, pump aerosols or other devices known per se for liquid misting and solid atomizing which in particular permit an exact individual dosage.

Pharmaceutical compositions suitable for use in the invention include compositions wherein compounds for depleting mast cells, such as c-kit inhibitors, or compounds inhibiting mast cells degranulation are contained in an effective amount to achieve the intended purpose. The determination of an effective dose is well within the capability of those skilled in the art. A therapeutically effective dose refers to that amount of active ingredient, which ameliorates the symptoms or condition. Therapeutic efficacy and toxicity may be determined by standard pharmaceutical procedures in cell cultures or experimental animals, e.g., ED50 (the dose therapeutically effective in 50% of the population) and LD50 (the dose lethal to 50% of the population). The dose ratio of toxic to therapeutic effects is the therapeutic index, and it can be expressed as the ratio, LD50/ED50. Pharmaceutical compositions which exhibit large therapeutic indices are preferred.

### Example 1

### Mast Cell Inhibitor as Therapy Against Sulfur-Mustard

The acute phase of the skin inflammation after exposure to sulfur mustard is partly due to the release by mast cells of mediators such as histamine and PGE2. These mediators are well known to induce a vasodilatation leading to the recruitment on the inflammation site of cells, being responsible for the secondary inflammatory reaction (T lymphocytes, neutrophils, macrophages). In addition, mast cells, when activated, release also other mediators and in particular LTC4, which has a chemotactic effect on neutrophils, cytokines (TNF-α, IL-6, GM-CSF) which activate inflammatory cells, holding the inflammatory process.

Besides, activated mast cells release chemokines such as IL-8 and TNF-α, which increase the recruitment on the inflammatory site of neutrophils and macrophages, that in turn secrete proteolytic enzymes and a myriad of cytokines including TNF-α, that amplify the inflammatory response and the damages to the tissues.

We have in hands a number of potent anti-MC agents as described above. These anti-MC agents act through inhibition of c-Kit leading thus not only to inhibition of mast cell survival but also to the one of mast cell activation.

A treatment with a MC inhibitor following exposure to sulfur mustard induces a decrease in the activation of mast cells. This decrease in the MC activation results in a reduction in the secretion of histamine, leucotrienes, cytokines and chemokines, limiting the activation and recruitment of neutrophils and macrophages.

### Example 2

### Mast Cell Inhibitor as Therapy Against Anthrax Toxin

The three major problems of clinical management of patients suffering from Anthrax, and particularly from its pulmonary or systemic forms (ie the forms are accompanied by a significant mortality and morbidity) are the following:

First, although most of the bacillus anthracis strains are not resistant to most of the antibiotics used to care patients, it does not appear impossible to manufacture resistant strains that could be used as biological weapon.

Second, the major pathogenic mechanism involved in Anthrax infection is not the growth of the bacillus anthracis but the synthesis and release of Anthrax toxin that is responsible for morbidity and mortality and against which there is no antidote.

Third, the extension of the infection into the body remains without clinical signs until the amount of Anthrax toxin released has caused irreversible tissue damages. At this time antibiotic therapy might not be able to counteract these pro-inflammatory related tissue damages.

Several bacteria secrete products, and particularly toxins, that activate MCs and evoke a mediator response that is different from their normal response to activation. For example, bacterial toxins can sometimes induce the hypersecretion of inflammatory mediators by MCs, leading to detrimental effects for the host. Thus, whereas proinflammatory mediators of MCs, such as TNF-α and superoxide anions, are beneficial to the host because they recruit neutrophils and are bactericidal, respectively, the same mediators, when released in excessive amounts or at inappropriate times, might cause marked pathological effects to the surrounding tissue, such as edema, necrosis and fibrosis.

Pure populations of rodent or human MCs are routinely obtained in our laboratory by culture of hematopoietic progenitors in the presence of appropriate growth factors. Anthrax toxins effect can tested by the release of their mediators, and particularly of TNF-α. A second set of experiments, conducted in vivo, with the help of MC-deficient W/Wv mice, wild-type mice or MC-reconstituted W/Wv mice, is to demonstrate that MC-derived TNF-α is a critical factor for the morbidity and the mortality induced by sub-lethal or lethal injection of Anthrax toxins, respectively. A third set of experiments, is to show in vivo, on mice depleted of mast cells by injection of a AB compound as depicted above, that this depletion induces a protection of the animals against the morbidity and the mortality induced by sub-lethal or lethal injection of Anthrax toxins, respectively.

AB compounds of formula III, IV, V and X are selective and potent c-Kit and mast cell inhibitors. The specific compounds as lists above are non limitative illustrative examples of AB compounds. They display IC50 below 5 μM, 1 μM or even 0.1 μM on different forms of c-KIT.

Of interest, in rodent models as well as in human the activation of the c-kit receptor is critical for MC survival and interferes also during the process of MC activation. Thus, the AB compound induces both in vitro and in vivo the depletion of MC population. This has been demonstrated using a model of in vitro derived primary human or mouse MC (FIG. 2) and a model of in vivo administration in mice.

### Example 3

### Inhibition of Enzymatic Kinase Activity of Purified c-Kit by a AB Compound as Defined Above

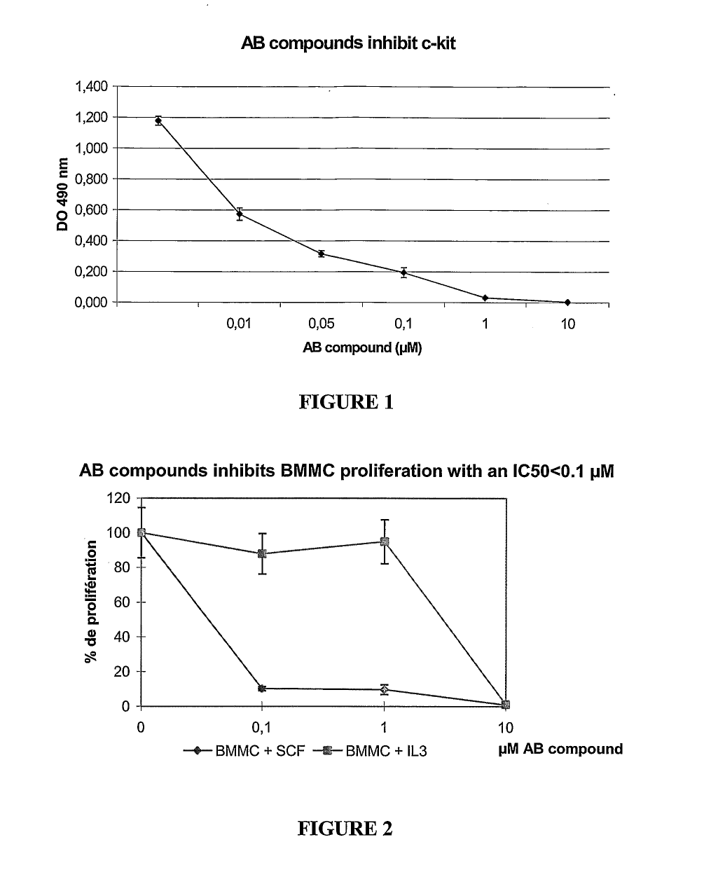

Of interest, we have shown that the AB compound potently inhibited the enzymatic activity of the c-Kit protein tyrosine kinase with an IC50=0.01 μM. These data are shown in FIG. 1 (in vitro inhibition of the catalytic domain of c-Kit (JM and WT) tyrosine kinase by the AB compound).

In these experiments, the AB compound was assayed in vitro for inhibition of c-kit tyrosine kinase activity. Experiments were performed using purified intracellular domain of c-kit expressed in baculovirus. The evaluation of the kinase activity was assessed by the phosphorylation of a tyrosine containing target peptide measured with “in house” established ELISA assay. Results obtained demonstrate that the AB compound inhibited the tyrosine kinase activity of c-Kit with an IC50 of 0.01 μM. Further experiments (data not shown) indicate that the AB compound acts as perfect competitive inhibitor of ATP.

Of note, although the mechanism of action of the AB compound is a perfect competition with ATP for binding to the kinase, our compound display a remarkable specificity towards c-kit. Indeed the AB compound has been tested in in vitro assays on several putative kinase targets and data from these experiments (Table 1) clearly show that the AB compound is a selective inhibitor of c-Kit.

TABLE 1
Inhibition of various protein tyrosine
kinases by the AB compound in vitro

| Enzyme/Cell line | |
|---|---|
| In vitro enzymatic assay on purified kinases | IC50 [μM] |
| c-Kit | 0.01 |
| PDGF-beta | 0.49 |
| ABL1 | 5.7 |
| VEGFR1 | IC50 > 100 |
| EGFR | IC50 > 100 |
| FGFR1 | IC50 > 100 |
| FLT3 | IC50 > 100 |
| JAK2 | IC50 > 100 |
| AKT1 | 57 |
| PKC-alpha | 100 |
| SRC | IC50 > 100 |
| IGF1R | IC50 > 100 |
| PM1 | 19 |

### Example 4

### Inhibitory Activity of the AB Compound on Murine Primary Mast Cells Proliferation

In order to show that the AB compound is able to inhibit the survival and proliferation of MC, we tested this compound on the proliferation of primary MC cultures obtained from murine bone marrow cells grown and expanded in Mast Cell Medium (MCM) supplemented with 12.5 ng/ml recombinant murine IL3. The inhibitory effect of the AB compound was then assessed on these cells that were seeded at 10⁴ cells per well in a 96 wells plates in the presence of either IL3 (control) or SCF. Cells were treated for 48 hours at 37° C. with various concentration of the AB compound and proliferation was monitored using WST-1 reagent from Roche diagnostic.

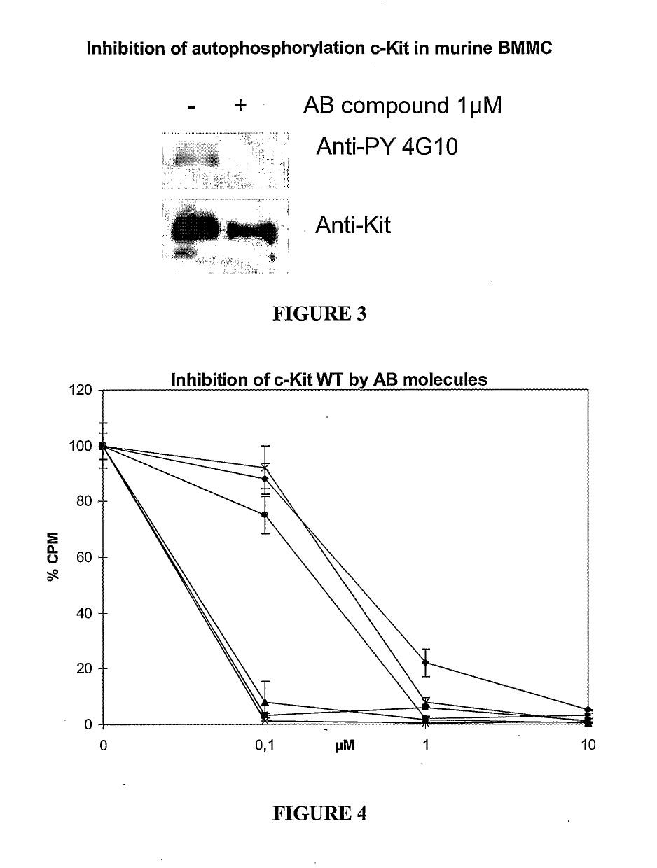

As shown in FIG. 2 the inhibition of the growth of this MC culture in the presence of IL-3 was observed at concentration higher than 1 μM of the AB compound. By contrast, the AB compound potently and dose-dependently inhibited the growth of the same MC when they were cultured in the presence of SCF (with an IC50 of <0.1 μM). Again these in vitro data confirmed the potent and selective inhibitory activity of c-Kit tyrosine kinase activity as well as the ability of the AB compound to inhibit almost completely the survival of MC population at concentration lower than 0.1 μM. In addition experiments done on the same cells showed that the AB compound induced a complete inhibition of tyrosine phosphorylation of c-Kit in MC treated with SCF (FIG. 3).

0.5 10⁶ cells were treated with 1 μM AB compound for 2 hours or left untreated before stimulation with SCF for 5 min. The cells were then processed for western blot analysis using an anti-phosphotyrosine antibody.

### Example 5

### The AB Compound is Able to Deplete Normal Mice from Mast Cells and is a Successful Preclinical Molecule

In order to demonstrate that the AB compound is able to decrease selectively the number of MCs present in normal mice, batches of 12 C57BL mice have been treated daily with non-toxic and efficient concentrations of AB60 for 7, 10, 22 or 29 days. AB60 (12.5, 25 or 50 mg/kg) will be administered by intraperitoneal injections. Control, vehicle alone treated batches of animals have also been constituted. At the end of each time of treatment, and for each dose, mice have been sacrificed and the number of MC have been analyzed in various tissues (peritoneal fluid, muscle, etc), and compared with that of control animals. Data from this experiment clearly showed that the AB compound depleted selectively, in a dose and time-dependent manner, MCs in normal mouse models. For instance, at 25 mg/kg, the AB compound induced a complete disappearance of MC from peritoneal fluids (mean value: 0+/−0%) after an administration of 10 days (value for control mice at 10 days: 3.5+/−1.5 of MC in peritoneal fluids).

The AB compound has successfully completed preclinical development in September 2003. Safety pharmacology studies revealed no significant effects of the AB compound on the central nervous, cardiovascular and respiratory systems.

The nonclinical potential toxicity of the AB compound has been tested in rats and dogs in single dose and repeat dose studies. Taking into consideration the minimal clinical findings observed in animals given 15 mg/kg/day and the reversibility of the findings, the AB60 oral NOAEL was established at 15 mg/kg/day in rats and in dogs.

The AB compound is currently manufactured under GMP conditions and 25 kilograms are being prepared for clinical development.

Preclinical studies have thus shown that the AB compound is a safe molecule that could be used in vivo.

The AB compound has clearly demonstrated its potent activity against c-Kit and mast cells both in vitro and in vivo, and a very slight if it exist toxicity in vivo. We have also developed other compounds of the above described family of molecules in order to find one or several compounds more potent and selective. At this time, several leads have demonstrated in in vitro models a more potent efficacy to block selectively wild-type (WT) c-Kit activity (FIG. 4).

1. A method for treating patients exposed to chemical or biological weapons, comprising administering a compound capable of depleting mast cells or a compound inhibiting mast cells degranulation in a human in need of such treatment.

2. The method according to claim 1 for treating patients suffering from exposure to chemical or biological toxic or lethal entities, comprising administering a c-kit inhibitor to a human in need of such treatment.

3. The method according to claim 2, wherein said c-kit inhibitor is a non-toxic, selective and potent c-kit inhibitor wherein it is unable to promote death of IL-3 dependent cells cultured in presence of IL-3.

4. The method according to claim 1 or 3 wherein said inhibitor is selected from the group consisting of:
2-(3-Substitutedaryl)amino-4-aryl-thiazoles such as 2-(3-amino)arylamino-4-aryl-thiazoles,
2-aminoaryloxazoles,
pyrimidine derivatives, more particularly N-phenyl-2-pyrimidine-amine derivatives,
indolinone derivatives, more particularly pyrrol-substituted indolinones,
monocyclic, bicyclic aryl and heteroaryl compounds,
and quinazoline derivatives.

5. The method according to claim 4, wherein said c-kit inhibitor is selected from compounds belonging to the 2-(3-Substitutedaryl)amino-4-aryl-thiazoles of formula III: 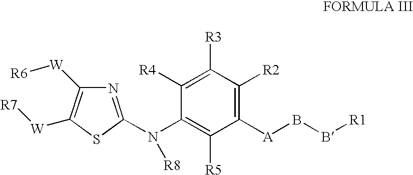
wherein
R⁶ and R⁷ are independently from each other chosen from one of the following:
i) hydrogen, a halogen (selected from F, Cl, Br or I),
ii) an alkyl¹ group defined as a linear, branched or cycloalkyl group containing from 1 to 10 carbon atoms, or from 2 or 3 to 10 carbon atoms, (for example methyl, ethyl, propyl, butyl, pentyl, hexyl . . . ) and optionally substituted with one or more hetereoatoms such as halogen (selected from F, Cl, Br or I), oxygen, and nitrogen (the latter optionally in the form of a pendant basic nitrogen functionality); as well as trifluoromethyl, carboxyl, cyano, nitro, formyl;
(iii) an aryl¹ group defined as phenyl or a substituted variant thereof bearing any combination, at any one ring position, of one or more substituents such as
halogen (selected from I, F, Cl or Br);
an alkyl¹ group;
a cycloalkyl, aryl or heteroaryl group optionally substituted by a pendant basic nitrogen functionality;
trifluoromethyl, O-alkyl¹, carboxyl, cyano, nitro, formyl, hydroxy, NH-alkyl¹, N(alkyl¹)(alkyl¹), and amino, the latter nitrogen substituents optionally in the form of a basic nitrogen functionality;
(iv) a heteroaryl¹ group defined as a pyridyl, pyrimidinyl, pyrazinyl, pyridazinyl, thienyl, thiazolyl, imidazolyl, pyrazolyl, pyrrolyl, furanyl, oxazolyl, isoxazolyl, triazolyl, tetrazolyl, indolyl, benzimidazole, quinolinyl group, which may additionally bear any combination, at any one ring position, of one or more substituents such as
halogen (selected from F, Cl, Br or I);
an alkyl¹ group;
a cycloalkyl, aryl or heteroaryl group optionally substituted by a pendant basic nitrogen functionality,
trifluoromethyl, O-alkyl¹, carboxyl, cyano, nitro, formyl, hydroxy, NH-alkyl¹, N(alkyl¹)(alkyl¹), and amino, the latter nitrogen substituents optionally in the form of a basic nitrogen functionality;
(v) trifluoromethyl, carboxyl, cyano, nitro, formyl, hydroxy, N(alkyl¹)(alkyl¹), and amino, the latter nitrogen substituents optionally in the form of a basic nitrogen functionality.
R⁸ is one of the following:
(i) hydrogen, or
(ii) a linear or branched alkyl group containing from 1 to 10 carbon atoms and optionally substituted with one or more hetereoatoms such as halogen (selected from F, Cl, Br or I), oxygen, and nitrogen, the latter optionally in the form of a pendant basic nitrogen functionality, or
(iii) CO—R8 or COOR8 or CONHR8 or SO2R8 wherein R8 may be
a linear or branched alkyl group containing from 1 to 10 carbon atoms and optionally substituted with one or more hetereoatoms such as halogen (selected from F, Cl, Br or I), oxygen, and nitrogen, the latter optionally in the form of a pendant basic nitrogen functionality, or
an aryl group such as phenyl or a substituted variant thereof bearing any combination, at any one ring position, of one or more substituents such as halogen (selected from F, Cl, Br or I), alkyl groups containing from 1 to 10 carbon atoms and optionally substituted with one or more hetereoatoms such as halogen (selected from F, Cl, Br or I), oxygen, and nitrogen, the latter optionally in the form of a pendant basic nitrogen functionality; as well as trifluoromethyl, C₁₋₆alkyloxy, carboxyl, cyano, nitro, formyl, hydroxy, C₁₋₆alkylamino, di(C₁₋₆alkyl)amino, and amino, the latter nitrogen substituents optionally in the form of a pendant basic nitrogen functionality; as well as CO—R, COO—R, CONH—R, SO2-R, and SO2NH—R wherein R is a linear or branched alkyl group containing from 1 to 10 carbon atoms and optionally substituted with at least one heteroatom, notably a halogen (selected from F, Cl, Br or I), oxygen, and nitrogen, the latter optionally in the form of a pendant basic nitrogen functionality, or
a heteroaryl group such as a pyridyl, pyrimidinyl, pyrazinyl, pyridazinyl, thienyl, thiazolyl, imidazolyl, pyrazolyl, pyrrolyl, furanyl, oxazolyl, isoxazolyl, triazolyl, tetrazolyl, indolyl, benzimidazole, quinolinyl group, which may additionally bear any combination, at any one ring position, of one or more substituents such as halogen (selected from F, Cl, Br or I), alkyl groups containing from 1 to 10 carbon atoms and optionally substituted with one or more hetereoatoms such as halogen (selected from F, Cl, Br or I), oxygen, and nitrogen, the latter optionally in the form of a pendant basic nitrogen functionality; as well as trifluoromethyl, C₁₋₆alkyloxy, carboxyl, cyano, nitro, formyl, hydroxy, C₁₋₆alkylamino, di(C₁₋₆alkyl)amino, and amino, the latter nitrogen substituents optionally in the form of a basic nitrogen functionality; as well as CO—R, COO—R, CONH—R, SO2-R, and SO2NH—R wherein R is a linear or branched alkyl group containing from 1 to 10 carbon atoms and optionally substituted with at least one heteroatom, notably a halogen (selected from F, Cl, Br or I), oxygen, and nitrogen, the latter optionally in the form of a pendant basic nitrogen functionality.
R2, R3, R4 and R5 each independently are selected from hydrogen, halogen (selected from F, Cl, Br or I), a linear or branched alkyl group containing from 1 to 10 carbon atoms and optionally substituted with one or more hetereoatoms such as halogen (selected from F, Cl, Br or I), oxygen, and nitrogen, the latter optionally in the form of a pendant basic nitrogen functionality; as well as trifluoromethyl, C₁₋₆alkyloxy, amino, C₁₋₆alkylamino, di(C₁₋₆alkyl)amino, carboxyl, cyano, nitro, formyl, hydroxy, and CO—R, COO—R, CONH—R, SO2-R, and SO2NH—R wherein R is a linear or branched alkyl group containing from 1 to 10 carbon atoms and optionally substituted with at least one heteroatom, notably a halogen (selected from F, Cl, Br or I), oxygen, and nitrogen, the latter optionally in the form of a pendant basic nitrogen functionality.
A is: CH2, O, S, SO2, CO, or COO,
B is a bond or NH, NCH3, NR*, (CH2)n (n is 0, 1 or 2), O, S, SO2, CO, or COO,
B′ is a bond or NH, NCH3, NR*, (CH2)n (n is 0, 1 or 2), O, S, SO2, CO or COO;
R* being an alkyl¹, aryl¹ or heteroaryl¹
W is a bond or a linker selected from NH, NHCO, NHCOO, NHCONH, NHSO2, NHSO2NH, CO, CONH, COO, COCH2, (CH2)n (n is 0, 1 or 2), CH2-CO, CH2COO, CH2-NH, O, OCH2, S, SO2, and SO2NH
R¹ is:
a) a linear or branched alkyl group containing from 1 to 10 carbon atoms optionally substituted with at least one heteroatom, notably a halogen selected from I, Cl, Br and F, and/or bearing a pendant basic nitrogen functionality;
b) an aryl or heteroaryl group optionally substituted by an alkyl or aryl group optionally substituted with a heteroatom, notably a halogen selected from I, Cl, Br and F or bearing a pendant basic nitrogen functionality
c) an alkyl¹, aryl¹ or heteroaryl¹.

6. A method according to claim 5, wherein said c-kit inhibitor is selected from compounds of formula V: 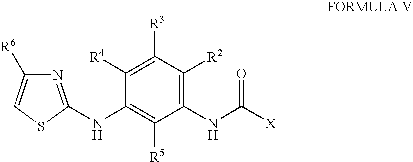
wherein X is R or NRR′ and wherein R and R′ are independently chosen from H, an aryl, a heteroaryl, an alkyl, or a cycloalkyl group optionally substituted with at least one heteroatom, such as for example a halogen chosen from F, I, Cl and Br and optionally bearing a pendant basic nitrogen functionality; or an aryl, a heteroaryl, an alkyl or a cycloalkyl group substituted with an aryl, a heteroaryl, an alkyl or a cycloalkyl group optionally substituted with at least one heteroatom, such as for example a halogen chosen from F, I, Cl and Br and optionally bearing a pendant basic nitrogen functionality,
R² is hydrogen, halogen or a linear or branched alkyl group containing from 1 to 10 carbon atoms, trifluoromethyl or alkoxy;
R³ is hydrogen, halogen or a linear or branched alkyl group containing from 1 to 10 carbon atoms, trifluoromethyl or alkoxy;
R⁴ is hydrogen, halogen or a linear or branched alkyl group containing from 1 to 10 carbon atoms, trifluoromethyl or alkoxy;
R⁵ is hydrogen, halogen or a linear or branched alkyl group containing from 1 to 10 carbon atoms, trifluoromethyl or alkoxy;
R⁶ is one of the following:
(i) an aryl group such as phenyl or a substituted variant thereof bearing any combination, at any one ring position, of one or more substituents such as halogen, alkyl groups containing from 1 to 10 carbon atoms, trifluoromethyl, and alkoxy;
(ii) a heteroaryl group such as a 2, 3, or 4-pyridyl group, which may additionally bear any combination of one or more substituents such as halogen, alkyl groups containing from 1 to 10 carbon atoms, trifluoromethyl and alkoxy;
(iii) a five-membered ring aromatic heterocyclic group such as for example 2-thienyl, 3-thienyl, 2-thiazolyl, 4-thiazolyl, 5-thiazolyl, which may additionally bear any combination of one or more substituents such as halogen, an alkyl group containing from 1 to 10 carbon atoms, trifluoromethyl, and alkoxy.
iv) H, a halogen selected from I, F, Cl or Br; NH2, NO2 or SO2-R, wherein R is a linear or branched alkyl group containing one or more group such as 1 to 10 carbon atoms, and optionally substituted with at least one heteroatom, notably a halogen selected from I, Cl, Br and F, and/or bearing a pendant basic nitrogen functionality.

7. The method according to claim 4, wherein said c-kit inhibitor is selected from 2-aminoaryloxazoles of formula X: 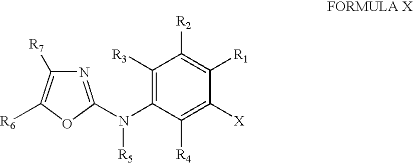
wherein substituents R1-R7 and X are defined as follows:
R1, R2, R3 and R4 each independently are selected from hydrogen, halogen (selected from F, Cl, Br or I), a linear or branched alkyl group containing from 1 to 10 carbon atoms and optionally substituted with one or more hetereoatoms such as halogen (selected from F, Cl, Br or I), oxygen, and nitrogen, the latter optionally in the form of a pendant basic nitrogen functionality; as well as trifluoromethyl, C₁₋₆alkyloxy, amino, C₁₋₆alkylamino, di(C₁₋₆alkyl)amino, carboxyl, cyano, nitro, formyl, hydroxy, and CO—R, COO—R, CONH—R, SO2-R, and SO2NH—R wherein R is a linear or branched alkyl group containing from 1 to 10 carbon atoms and optionally substituted with at least one heteroatom, notably a halogen (selected from F, Cl, Br or I), oxygen, and nitrogen, the latter optionally in the form of a pendant basic nitrogen functionality.
R5 is one of the following:
(i) hydrogen, or
(ii) a linear or branched alkyl group containing from 1 to 10 carbon atoms and optionally substituted with one or more hetereoatoms such as halogen (selected from F, Cl, Br or I), oxygen, and nitrogen, the latter optionally in the form of a pendant basic nitrogen functionality, or
(iii) C0-R8 or COOR8 or CONHR8 or SO2R8 wherein R8 may be
a linear or branched alkyl group containing from 1 to 10 carbon atoms and optionally substituted with one or more hetereoatoms such as halogen (selected from F, Cl, Br or I), oxygen, and nitrogen, the latter optionally in the form of a pendant basic nitrogen functionality, or
an aryl group such as phenyl or a substituted variant thereof bearing any combination, at any one ring position, of one or more substituents such as halogen (selected from F, Cl, Br or I), alkyl groups containing from 1 to 10 carbon atoms and optionally substituted with one or more hetereoatoms such as halogen (selected from F, Cl, Br or I), oxygen, and nitrogen, the latter optionally in the form of a pendant basic nitrogen functionality; as well as trifluoromethyl, C₁₋₆alkyloxy, carboxyl, cyano, nitro, formyl, hydroxy, C₁₋₆alkylamino, di(C₁₋₆alkyl)amino, and amino, the latter nitrogen substituents optionally in the form of a pendant basic nitrogen functionality; as well as CO—R, COO—R, CONH—R, SO2-R, and SO2NH—R wherein R is a linear or branched alkyl group containing from 1 to 10 carbon atoms and optionally substituted with at least one heteroatom, notably a halogen (selected from F, Cl, Br or I), oxygen, and nitrogen, the latter optionally in the form of a pendant basic nitrogen functionality, or
a heteroaryl group such as a pyridyl, pyrimidinyl, pyrazinyl, pyridazinyl, thienyl, thiazolyl, imidazolyl, pyrazolyl, pyrrolyl, furanyl, oxazolyl, isoxazolyl, triazolyl, tetrazolyl, indolyl, benzimidazole, quinolinyl group, which may additionally bear any combination, at any one ring position, of one or more substituents such as halogen (selected from F, Cl, Br or I), alkyl groups containing from 1 to 10 carbon atoms and optionally substituted with one or more hetereoatoms such as halogen (selected from F, Cl, Br or I), oxygen, and nitrogen, the latter optionally in the form of a pendant basic nitrogen functionality; as well as trifluoromethyl, C₁₋₆alkyloxy, carboxyl, cyano, nitro, formyl, hydroxy, C₁₋₆alkylamino, di(C₁₋₆alkyl)amino, and amino, the latter nitrogen substituents optionally in the form of a basic nitrogen functionality; as well as CO—R, COO—R, CONH—R, SO2-R, and SO2NH—R wherein R is a linear or branched alkyl group containing from 1 to 10 carbon atoms and optionally substituted with at least one heteroatom, notably a halogen (selected from F, Cl, Br or I), oxygen, and nitrogen, the latter optionally in the form of a pendant basic nitrogen functionality.
R6 and R7 each independently are selected from:
i) hydrogen, a halogen (selected from F, Cl, Br or I), or
ii) an alkyl¹ group defined as a linear, branched or cycloalkyl group containing from 1 to 10 carbon atoms and optionally substituted with one or more hetereoatoms such as halogen (selected from F, Cl, Br or I), oxygen, and nitrogen (the latter optionally in the form of a pendant basic nitrogen functionality); as well as trifluoromethyl, carboxyl, cyano, nitro, formyl; as well as CO—R, COO—R, CONH—R, SO2-R, and SO2NH—R wherein R is a linear or branched alkyl group containing 1 to 10 carbon atoms and optionally substituted with at least one heteroatom, notably a halogen (selected from F, Cl, Br or I), oxygen, and nitrogen, the latter optionally in the form of a pendant basic nitrogen functionality; as well as a cycloalkyl or aryl or heteroaryl group optionally substituted by a pendant basic nitrogen functionality, or
(iii) an aryl¹ group defined as phenyl or a substituted variant thereof bearing any combination, at any one ring position, of one or more substituents such as
halogen (selected from I, F, Cl or Br);
an alkyl group;
a cycloalkyl, aryl or heteroaryl group optionally substituted by a pendant basic nitrogen functionality;
trifluoromethyl, O-alkyl¹, carboxyl, cyano, nitro, formyl, hydroxy, NH-alkyl¹, N(alkyl¹)(alkyl¹), and amino, the latter nitrogen substituents optionally in the form of a basic nitrogen functionality;
NHCO—R or NHCOO—R or NHCONH—R or NHSO2-R or NHSO2NH—R or CO—R or COO—R or CONH—R or SO2-R or SO2NH—R wherein R corresponds to hydrogen, alkyl¹, aryl or heteroaryl, or
(iv) a heteroaryl¹ group defined as a pyridyl, pyrimidinyl, pyrazinyl, pyridazinyl, thienyl, thiazolyl, imidazolyl, pyrazolyl, pyrrolyl, furanyl, oxazolyl, isoxazolyl, triazolyl, tetrazolyl, indolyl, benzimidazole, quinolinyl group, which may additionally bear any combination, at any one ring position, of one or more substituents such as
halogen (selected from F, Cl, Br or I);
an alkyl¹ group;
a cycloalkyl, aryl or heteroaryl group optionally substituted by a pendant basic nitrogen functionality,
trifluoromethyl, O-alkyl¹, carboxyl, cyano, nitro, formyl, hydroxy, NH-alkyl¹, N(alkyl¹)(alkyl¹), and amino, the latter nitrogen substituents optionally in the form of a basic nitrogen functionality;
NHCO—R or NHCOO—R or NHCONH—R or NHSO2-R or NHSO2NH—R or CO—R or COO—R or CONH—R or SO2-R or SO2NH—R wherein R corresponds to hydrogen, alkyl¹, or
(v) an O-aryl¹, or NH-aryl¹, or O-heteroaryl¹ or NH-heteroaryl¹ group
(vi) trifluoromethyl, O-alkyl¹, carboxyl, cyano, nitro, formyl, hydroxy, NH-alkyl¹, N(alkyl¹)(alkyl¹), and amino, the latter nitrogen substituents optionally in the form of a basic nitrogen functionality, or
(vi) NHCO—R or NHCOO—R or NHCONH—R or NHSO2-R or NHSO2NH—R or CO—R or COO—R or CONH—R or SO2-R or SO2NH—R wherein R corresponds to hydrogen, alkyl¹, aryl or heteroaryl.
X is:
—NR9R10, wherein R9 and/or R10 are hydrogen or:
i) an alkyl group, CF3 or
ii) an aryl¹, heteroaryl¹ or cycloalkyl group optionally substituted by a pendant basic nitrogen functionality, or
iii) a CO—R, COO—R, CON—RR′ or SO2-R, where R and R′ are a hydrogen, alkyl¹, aryl¹ or heteroaryl¹, optionally substituted by a pendant basic nitrogen functionality;
or:
—CO—NR9R10, wherein R9 and/or R10 are hydrogen or:
i) an alkyl¹ group, CF3 or
ii) an aryl¹, heteroaryl¹ or cycloalkyl group optionally substituted by a pendant basic nitrogen functionality.

8. The method according to claim 4, wherein said inhibitor is selected from the group consisting of N-phenyl-2-pyrimidine-amine derivatives having the formula II: 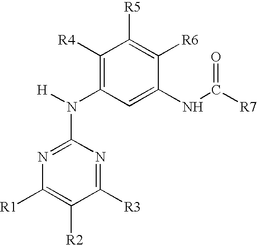
wherein R1, R2 and R3 are independently chosen from H, F, Cl, Br, I, a C1-C5 alkyl or a cyclic or heterocyclic group, especially a pyridyl group;
R4, R5 and R6 are independently chosen from H, F, Cl, Br, I, a C1-C5 alkyl, especially a methyl group;
and R7 is a phenyl group bearing at least one substituent, which in turn possesses at least one basic site, such as an amino function.

9. The method according to claim 8, wherein said inhibitor is the 4-(4-méthylpipérazine-1-ylméthyl)-N-[4-méthyl-3-(4-pyridine-3-yl)pyrimidine-2 ylamino)phényl]-benzamide.

10. A method for treating patients exposed to chemical or biological weapons comprising administering to a human in need of such treatment a compound that is a selective, potent and non toxic inhibitor of activated c-kit obtainable by a screening method which comprises:
a) bringing into contact (i) activated c-kit and (ii) at least one compound to be tested; under conditions allowing the components (i) and (ii) to form a complex,
b) selecting compounds that inhibit activated c-kit,
c) testing and selecting a subset of compounds identified in step b), which are unable to promote death of IL-3 dependent cells cultured in presence of IL-3.

11. The method according to one of claims 1 to 10 for treating of all forms of human immunodeficiency virus type 1, type 2, for treating accidental, terrorist or war exposure to different chemical or biological toxic or lethal entities, including bacterial toxins, hydrocarbons, pesticides, heavy metal, vesicants, organochlorine agents, alkylating agents, for example sulfur mustard (2,2′-dichlorodiethyl sulfide; SM or HD) and derivatives thereof, nerve agents, blister agents and Bacillus anthracis (Anthrax).

12. The use of a compound as defined in one of claims 1 to 9 or obtainable by the method of claim 10 to manufacture a medicament for treating accidental, terrorist or war exposure to different chemical or biological toxic or lethal entities, including bacterial toxins, hydrocarbons, pesticides, heavy metal, vesicants, organochlorine agents, alkylating agents, for example sulfur mustard (2,2′-dichlorodiethyl sulfide; SM or HD) and derivatives thereof, nerve agents, blister agents and Bacillus anthracis (Anthrax).
